(19) United States
(12) Patent Application Publication
(10) Pub. No.: US 2019/0046104 A1
(43) Pub. Date: Feb. 14, 2019

(54) ELECTRICAL IMPEDANCE IMAGING

(71) Applicant: THE UNIVERSITY OF WESTERN ONTARIO, London (CA)

(72) Inventors: Abbas SAMANI, London (CA); Seyyed HESABGAR, London (CA); David HOLDSWORTH, London (CA); Ravi MENON, London (CA)

(21) Appl. No.: 15/760,165

(22) PCT Filed: Sep. 14, 2016
(86) PCT No.: PCT/CA2016/051084

§ 371 Date: Mar. 14, 2018

Related U.S. Application Data
(60) Provisional application No. 62/218,984, filed on Sep. 15, 2015.

(51) Int. Cl. A61B 5/00; A61B 5/053; G01R 27/26

(52) U.S. Cl. CPC A61B 5/4312; G01R 27/26; A61B 5/0536

(57) ABSTRACT

An electrical impedance scanner includes a first planar plate, which includes a number of excitation cells; and a second planar plate, which includes a number of detector cells. The first planar plate is held in spaced parallel relation to the second planar plate, such that a a chamber is defined. The first and second planar plates are varranged to align each excitation cell with a corresponding detector cell in a one-to-one paired relationship. Each paired excitation cell and detector cell is configured for synchronized activation with an electric field. Systems can incorporate the scanner and methods relate to use of the scanner.

### BACKGROUND OF THE INVENTION

### Field of the Invention

The present invention relates to electrical impedance imaging, and more particularly to electrical impedance imaging for medical applications.

### Description of the Related Art

Many current medical imaging methods have limitations such as tissue ionization, noise and high cost, which may impact their effectiveness in the clinic. For instance, X-ray and Computer Tomography (CT) imaging techniques, which are based on tissue attenuation coefficient, both expose patients to radiation and also are not capable of generating images with high contrast for many soft tissue regions. In contrast to X-ray and CT, MRI does not involve exposure to radiation, but is expensive and often requires contrast agents for imaging tissues. Another common imaging modality is ultrasound which visualizes tissue acoustic properties. This modality often suffers from high levels of noise, frequently leading to low quality imaging. In addition to these limitations, it is known that various imaging modalities display only specific types of data (e.g. morphology, microcalcification, etc.) pertaining to tissue pathology. As such, clinicians often use the approach of fusing data obtained from different modalities for more accurate diagnosis.

Imaging techniques are founded on tissue physical properties that are reconstructed by processing measured data using a mathematical framework which describes the physics of interaction between tissue and its excitation. The heterogeneity various tissues exhibit in terms of the physical property used in an imaging technique influences the medical image contrast in clinical applications, and affects the technique's sensitivity and specificity. Among tissue physical properties that have not been sufficiently explored for developing effective medical imaging techniques, electrical properties have good potential. While tissue electrical impedance (EI) has been somewhat explored for medical imaging, leading to the Electrical Impedance Tomography (EIT) technique, electrical permittivity (EP) or electrical capacitance (EC) have not been given significant attention in the medical imaging field. EI encompasses electrical resistance (R) and electrical capacitance (C). R is a function of EC and tissue distribution while C is a function of EP and tissue distribution. Unlike R and C, EC and EP are intrinsic electrical properties of the sample being analyzed.

While EIT has been developed and significantly improved over the past two decades, it still suffers from two major drawbacks which have limited its clinical utility. The first is that the range of EI variation for most biological tissues at low frequencies, i.e. 100 KHz or lower, is limited (S Gabriel, R W Lau and C Gabriel, The dielectric properties of biological tissues: III. Parametric models for the dielectric spectrum of tissues, Phys. Med. Biol. 41 (1996) 2271-2293. S Gabriel, R W Lau and C Gabriel, The dielectric properties of biological tissues: Measurements in the frequency range 10 Hz to 20 GHz, Phys. Med. Biol. 41 (1996) 2251-2269). This means that obtaining high contrast EI images at low frequencies is often not feasible. The second is that imaging tissue with EI requires the use of multiple independently positioned contacting electrodes. The number of electrodes may be five, six, seven, eight, or even more. However, in many clinical applications, contacting electrodes either cannot be used or using the required number of electrodes is impractical.

Accordingly, there is a continuing need for alternative medical imaging or medical screening techniques based on measurement of electrical properties.

### SUMMARY OF THE INVENTION

In an aspect there is provided an electrical impedance scanner, comprising:

a first planar plate comprising a plurality of excitation cells;

a second planar plate comprising a plurality of detector cells;

the first planar plate held in spaced parallel relation to the second planar plate and defining a chamber therebetween;

the first and second planar plates arranged to align each excitation cell with a corresponding detector cell in a one-to-one paired relationship; and

each paired excitation cell and detector cell configured for synchronized activation with an electric field communicating therebetween.

In further aspects, systems incorporating the scanner, and methods and computer readable medium relating to use of the scanner are also provided.

### BRIEF DESCRIPTION OF THE DRAWINGS

FIG. 1 shows a schematic view of a impedance scanner;

FIG. 2 shows a schematic cross-sectional view of a prior art impedance sensor;

FIG. 3 shows a 2D non-uniform electric field in a homogeneous medium within the prior art impedance sensor shown in FIG. 2;

FIG. 4 shows a computer controlled imaging system comprising the impedance scanner shown in FIG. 1;

FIG. 5 shows a schematic of a sample section of a phantom consisting of two tissues (e.g. background healthy tissue and tumor) placed between two-parallel-plates of a diaphragm variant of the impedance scanner shown in FIG. 1;

FIG. 6 shows block shape phantoms with cylindrical inclusions with various sizes mimicking tumor in healthy background tissue used in an in silico phantom study for permittivity image reconstruction using data back propagation;

FIG. 7 shows a tissue mimicking phantom consisting of background and inclusion with permittivity values of 180 F/m and 420 F/m, respectively;

FIG. 8 shows plots of deviation error from linear approximation vs. frequency along the centreline of in silico breast phantoms with 10, 15 and 25 mm diameter spherical inclusions with permittivity values three times higher than the background tissue permittivity;

FIG. 9 shows plots of deviation error from linear approximation vs. frequency along the centreline of in silico bone-muscle phantom for the 10, 15 and 25 mm diameter cylindrical inclusions with permittivity values twenty times lower than the background tissue permittivity;

FIG. 10 shows plots of deviation error from linear approximation along the diaphragm's motion axis in the in silico breast phantom consisting of a block with 15 mm, 20 mm and 25 mm diameter cylindrical inclusions with permittivity values three times higher than the background tissue permittivity;

FIG. 11 shows reconstructed tomography images of the block phantoms shown in FIG. 6 with 15, 20 and 25 mm inclusions (top row) and corresponding segmented images obtained with a threshold value of 2000 F/m (bottom row);

FIG. 12 shows a plot of an experimentally acquired projection along the X-axis of the tissue mimicking phantom shown in FIG. 7;

FIG. 13 shows plots of (A) average values and (B) maximum values of the metric Δc as a function of permittivity and plate separation (phantom height);

FIG. 14 shows a schematic of a impedance scanner plate with a guard surrounding each excitation cell;

FIG. 15 shows a schematic of a needle biopsy variant of the impedance scanner shown in FIG. 1;

FIG. 16 shows a schematic of a breast tissue sample held between two cylindrical-shaped electrodes (right) and its equivalent electrical circuit at low frequencies, for example less than 30 KHz (left);

FIG. 17 shows plots of electrical resistance, impedance and capacitance of the adipose tissue model at 10 Hz to 1 MHz while placed between: two electrodes (A) and two parallel plates of a variant of impedance scanner shown in FIG. 1 (B);

FIG. 18 a schematic of a variant of the impedance scanner shown in FIG. 1 comprising two conductive parallel plates where the breast is gently squeezed in between and the breast is discretized using a uniform grid size where unknown impedance values are assigned to each pixel;

FIG. 19 shows (A) FE mesh of an in silico breast phantom consisting of half a cylinder embedding an inclusion, and (B) top view of the breast phantom with the inclusion on the bottom right side to mimic the breast upper outer quadrant;

FIG. 20 shows from left to right, reconstructed impedance, resistance, capacitance and phase angle images of the in silico breast phantom where the inclusion is located at the centers of the phantom height's top third (1st row), middle third (3rd row) and bottom third (5th row); Also, from left to right, variations profile of the impedance, resistance, capacitance and phase angle along a section crossing the middle of the inclusion corresponding to the in silico breast phantom where the inclusion is located at the centers of the phantom height's top third (2nd row), middle third (4th row) and bottom third (6th row);

FIG. 21 shows top row from left to right, reconstructed impedance, resistance, capacitance and phase angle images obtained from a tissue mimicking breast phantom study; and bottom row from left to right, variation profiles of the impedance, resistance, capacitance and phase angle signals along the section crossing the inclusion; and

FIG. 22 shows a method of using the impedance scanner shown in FIG. 18.

### DETAILED DESCRIPTION OF PREFERRED EMBODIMENTS

In contrast to EI, EP of biological tissues has a broad range. For example, at 100 MHz, EP of biological tissues varies from 6 F/m for fat to 56.2 F/m for brain white matter, and to 98 F/m for the kidney. The difference in tissue EP becomes even more significant at lower frequencies, so that at 1 KHz the EP values of the aforementioned tissues are 24104 F/m, 69811 F/m and 212900 F/m, respectively. Therefore, it can be concluded that image contrast and hence quality in EP imaging is potentially high. Table 1 presents EP values of six different human tissues at 100 Hz, 100 KHz and 100 MHz (S Gabriel, R W Lau and C Gabriel, The dielectric properties of biological tissues: Ill. Parametric models for the dielectric spectrum of tissues, Phys. Med. Biol. 41 (1996) 2271-2293. S Gabriel, R W Lau and C Gabriel, The dielectric properties of biological tissues: II. Measurements in the frequency range 10 Hz to 20 GHz, Phys. Med. Biol. 41 (1996) 2251-2269). Table 1 shows that tissue EP values decrease significantly with higher frequencies. The significant variation observed in tissue EP while excited with different frequencies indicates an important potential advantage of EP imaging where excitation frequency may be determined/tuned for given anatomical sites to improve image contrast.

TABLE 1
Frequency dependent variation of Electrical Permittivity of human tissues

| | | Bone | | Brain | Brain | |
|---|---|---|---|---|---|---|
| Tissue name | Muscle | (Cortical) | Blood | (White m.) | (Grey m.) | Fat |
| ε @ 100 Hz | 9329000 | 5852.8 | 5259.8 | 1667700 | 3906100 | 457060 |
| ε @ 100 KHz | 8089 | 227.6 | 5120 | 2108 | 3221 | 92.89 |
| ε @ 100 MHz | 65.9 | 15.3 | 76.8 | 56.8 | 80.14 | 6.07 |

Another important advantage of imaging EP over EI is the possibility of image data acquisition through capacitance measurement. Impedance sensors usually consist of a number of electrodes or metal plates, and the electrical capacitance is usually estimated through measurement of the voltage and current that passes through them. Achieving high image resolution using impedance sensors with electrodes is not practical because of the small number of relatively large electrodes used for data acquisition. Electrodes are discrete elements attached to the skin. Given the size of electrodes it is not possible to place a large enough number of such electrodes to achieve high image resolution—for example although 16 to 32 electrodes are typically used for imaging a thorax, this number of electrodes still does not produce a high resolution image.

EI encompasses electrical resistance (R) and electrical capacitance (C). R is a function of EC and tissue distribution while C is a function of EP and tissue distribution. Unlike R and C, EC and EP are intrinsic electrical properties of the sample being analyzed.

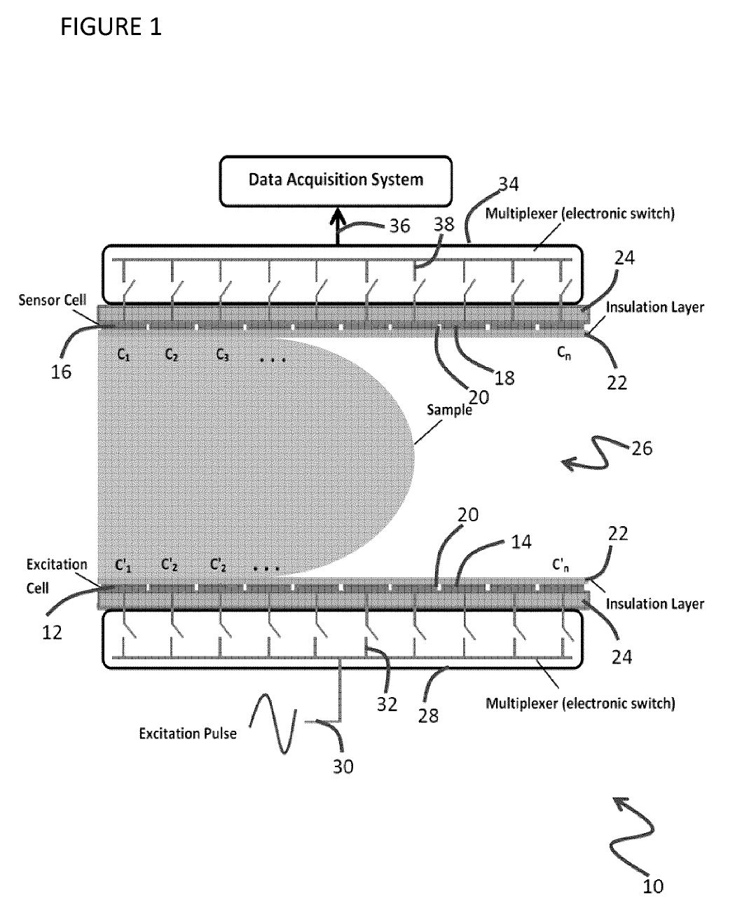

Now referring to the drawings, FIG. 1 shows an example of a impedance scanner 10 that can be used for medical impedance imaging including, for example, medical electrical permittivity imaging or impedance phase angle imaging. The impedance scanner 10 comprises two parallel planar plates, a first planar plate 12 housing a plurality of electrically conductive excitation cells 14 arranged in a first array and a second planar plate 16 housing a plurality of electrically conductive detector cells 18 arranged in a corresponding second array. Each excitation cell 14 is electrically isolated from other neighboring excitation cells by surrounding a perimeter of the excitation cell 14 with a non-conductive insulating gap 20. Each detector cell 18 is electrically isolated from other neighboring detector cells by surrounding a perimeter of the detector cell 18 with a non-conductive insulating gap 20. Thus, the first and second planar plates are segmented by the non-conductive insulating material 20, with each segment of the first planar plate including a single excitation cell and each segment of the second planar plate including a single detector cell.

Each of the first and second planar plates are bound by first and second surfaces with an insulation layer 22 covering the first surface and a grounding shield 24 covering the second surface. The first and second planar plates are arranged so that their respective insulation layers 22 face each other.

The first and second planar plates are maintained in a substantially parallel spaced relation defining a chamber 26 for receiving a biological sample in between the first and second planar plates. More specifically, the chamber 26 is defined in between the insulation layers 22 covering the first surfaces of the first and second planar plates. The insulation layers 22 provide contacting surfaces for the biological sample. The spacing between the first and second planar plates is adjustable so that surfaces of variously sized biological samples can be maintained in abutting contact with both the insulation layers 22 of the first and second planar plates.

The first and second planar plates are oriented so that an excitation cell and a corresponding detector cell are held in opposing alignment. When in use, each excitation cell and its corresponding detector cell are located on opposing sides of a biological sample. The plurality of excitation cells and the plurality of detector cells are typically equal in number so that each excitation cell opposes a detector cell in a one-to-one relationship (C′₁ to C₁, . . . , C′n to Cn). Each excitation cell and each detector cell can each be independently electrically controlled. A first multiplexer 28 comprises an input connector 30 in electrical communication with a voltage source and a plurality of relays, each relay 32 controlling electrical activation of a single excitation cell. The input connector 30 communicates an excitation signal from the voltage source through a closed relay to a corresponding excitation cell. The excitation signal may be modulated with respect to amplitude, frequency, or both amplitude and frequency. A second multiplexer 34 comprises an output connector 36 in electrical communication with data acquisition circuitry and a plurality of relays, each relay 38 controlling electrical communication of a single detector cell.

The first and second multiplexers function to synchronize any desired pattern of sequential activation or simultaneous activation of corresponding opposing pairs of excitation cells and detector cells to generate a substantially 1D uniform electric field traversing the chamber space through a biological sample, the substantially 1D uniform electric field having an orientation substantially perpendicular/normal to both the first and second planar plates. The data acquisition circuitry can measure an electrical property of a substantially 1D uniform electric field generated between each oppositely aligned pairing of a single excitation cell and a corresponding single detector cell. Typically, the measured electrical properties are the magnitude and phase angle of electrical impedance. Electrical resistance and electrical capacitance may be obtained from the impedance magnitude and phase angle. Electrical conductance and electrical permittivity may be obtained from electrical resistance and electrical capacitance, respectively.

The schematic impedance scanner has been validated experimentally. The following experimental examples are for illustration purposes only and are not intended to be a limiting description.

In a first set of experimental examples the impedance scanner is used to determine electrical permittivity (EP) of a sample held between the two parallel plates and process the EP data to generate an image of the sample.

Electrical permittivity (denoted by ε), is a parameter that shows how much electric field is generated per unit charge in a medium. It is usually measured through measuring electrical capacitance (C) as direct measurement of c may not be feasible. Electrical Capacitance (C) is a physical property of capacitors consisting of two conductors with a material (medium) between them and it can be measured using impedance scanners. It is a property of the capacitor which depends on the geometry of the conductors and the permittivity of the medium between them; it does not depend on the charge or potential difference between the conductors. The following is a fundamental relationship used to express C:

$$C = \frac{Q}{V} = \frac{∮ ɛ E \cdot ds}{\int E \cdot dl} \qquad ( 1 )$$

where Q is the electric charge, V the voltage between electrodes and E is the electric field. The surface integral in the numerator is carried out over the surface enclosing the conductor while the line integral in the denominator is calculated from the negative to positive conductor or low to high potential. As it can be seen from this relationship, if E is uniform, C will be proportional to the permittivity of medium between the electrodes or plates of the impedance scanner.

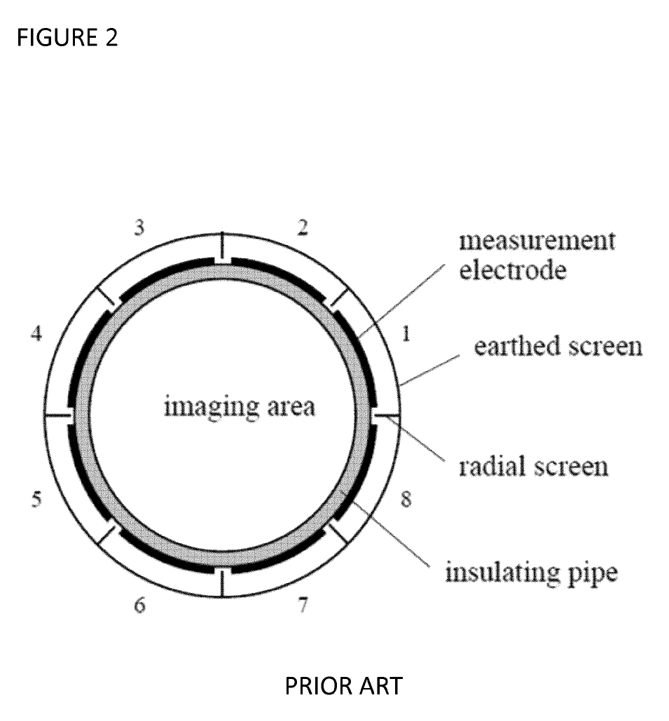

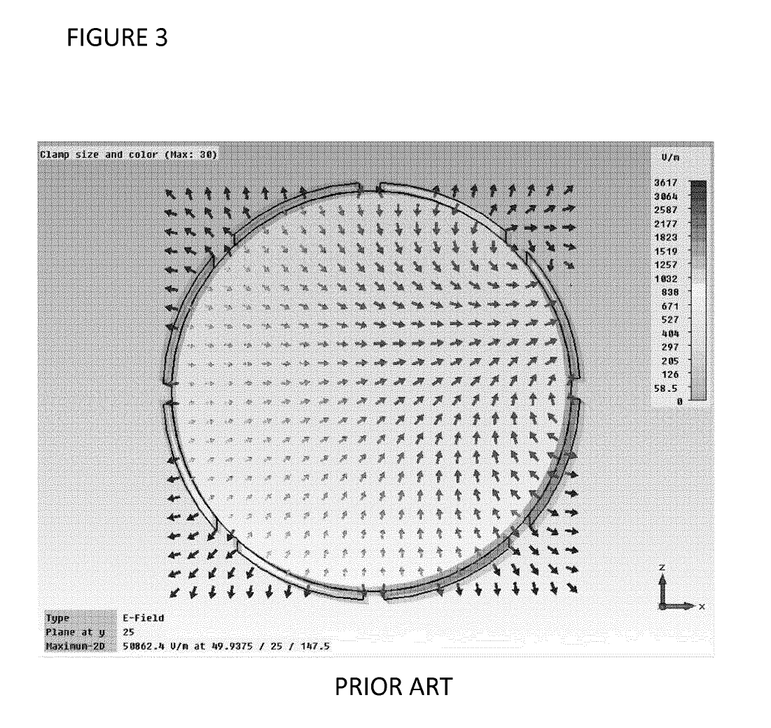

Most current Electrical Capacitance Tomography (ECT) systems have used a relatively simple electrode configuration with electrodes arranged around the periphery of the object being imaged. For data acquisition, one pair of the electrodes is activated at a time and the corresponding capacitance is measured. Another approach of medium excitation involves exciting one electrode with a positive potential while the other electrodes are activated with a negative voltage. For data acquisition, again the capacitance values between pairs of the positive electrode with each negative electrode are measured. FIG. 2 shows a typical configuration of a impedance sensor which is used by most researchers in the field, including, for example, Soleimani et al. (Manuchehr Soleimani, Phaneendra K. Yalavarthy, Hamid Dehghani; Helmholtz-Type Regularization Method for Permittivity Reconstruction Using Experimental Phantom Data of Electrical Capacitance Tomography; IEEE TRANSACTIONS ON INSTRUMENTATION AND MEASUREMENT, VOL. 59, NO. 1, January 2010), Alme et al. (Kjell Joar Alme, Saba Mylvaganam, Electrical Capacitance Tomography, Sensor Models, Design, Simulations, and Experimental Verification, IEEE SENSORS JOURNAL, VOL. 6, NO. 5, October 2006), Warsito et al. (Warsito Warsito, Qussai Marashdeh, Liang-Shih Fan, Electrical Capacitance Volume Tomography IEEE SENSORS JOURNAL, VOL. 7, NO. 4, April 2007) and Cao et al. (Zhang Cao, Lijun Xu, Wenru Fan, Huaxiang Wang, Electrical Capacitance Tomography for Sensors of Square Cross Sections Using Calderon's Method, IEEE TRANSACTIONS ON INSTRUMENTATION AND MEASUREMENT, VOL. 60, NO. 3, March 2011). A major issue with such impedance sensors is that the electric field inside the sensor between pairs of electrodes is neither uniform nor 1-dimensional, leading to a nonlinear relationship between the measured capacitance and medium permittivity distribution. A typical electric field developed in such sensors can be obtained using computational simulation and is depicted in FIG. 3. This field was created within a homogeneous medium (imaging area) which has uniformly distributed permittivity (ε) values of 1. Inhomogeneous media is expected to create a more complex electric field. As the electric field in these sensors is dependent on the permittivity distribution, it is not possible to derive an explicit expression which relates permittivity distribution to the measured capacitance. As such, previous studies have developed complex iterative inverse finite-element solutions to reconstruct the medium's permittivity using measured sensor's capacitance data. Apart from high computer power and time demand, such solutions suffer from serious ill-conditioning and uniqueness issues.

In contrast to prior art impedance sensors, the impedance scanner shown in FIG. 1 produces a sufficiently uniform electric field within the medium (e.g. tissue) to facilitate straight forward image reconstruction using linear equations, such as linear back projection. While electrical permittivity (EP) is an intrinsic property of a material, the electric field is a function of the geometry and permittivity distribution of the object being imaged and the scanner's configuration and excitation scheme. The latter two can be designed in order to achieve a linear electric field.

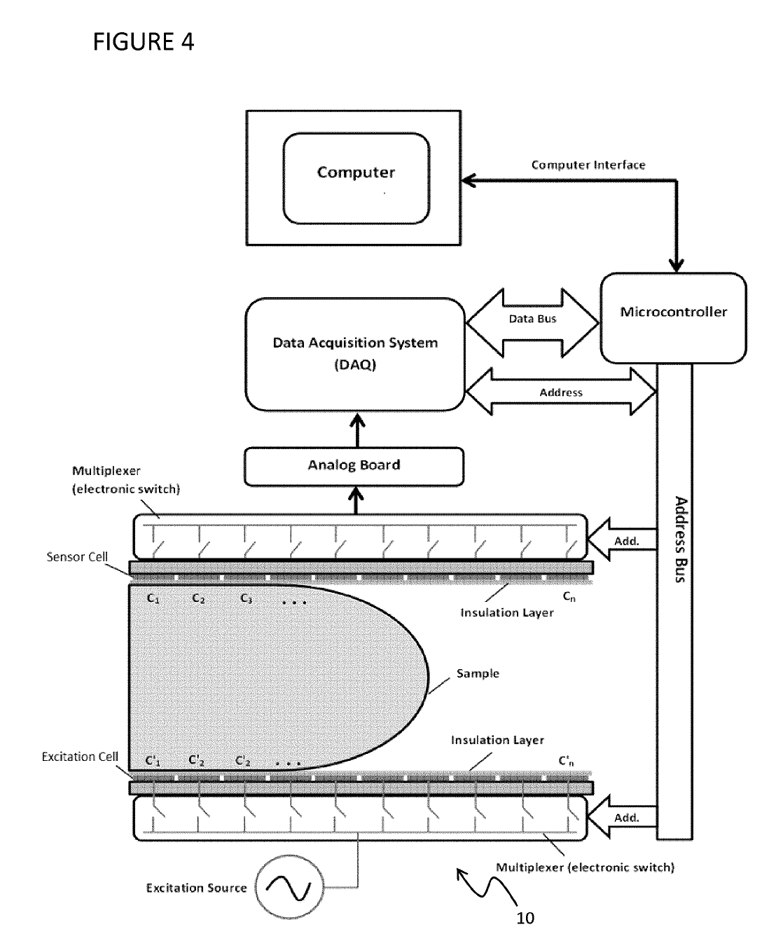

FIG. 4 shows the impedance scanner from FIG. 1 incorporated within a computer implemented imaging system. The scanner, consistent with FIG. 1, comprises two parallel plates housing opposing excitation cells and detector cells. The imaging system includes the parallel plate impedance scanner, multiplexers, analog board, data acquisition system (DAQ), microcontroller, data bus, address bus, computer interface and a computer. The microcontroller controls the performance of the whole system by providing proper addresses and control commands to the DAQ system and multiplexers via the address and data buses. It also communicates with the DAQ system via these buses to receive the A/D convergence data. Generation of A/D convergence data starts with feeding the electric current that passes through the tissue sample into an analog to digital (A/D) converter electronic chip. The A/D chip, measures the magnitude and phase angle of the input analog signal (i.e. electric current) by comparing it with a reference signal and converts the measured analog quantities (magnitude and phase angle) into binary codes. After reading the convergence data from the DAQ system, the microcontroller sends this information to the computer via a serial interface. The convergence data can then be processed using an image reconstruction computer code leading to the image. The image reconstruction computer code can easily be varied to accommodate different imaging techniques described herein including, for example, image reconstruction computer code based on resistance, conductivity, capacitance, permittivity, phase angle or any combination thereof. In order to switch the electronic relays inside the multiplexers, the microcontroller changes the address from 0 to n−1 on the address bus.

One option for an excitation/data acquisition scheme is that each pair of excitation cell and a corresponding opposite detector cell (e.g. C1 and C′1) is switched on and then off one at a time such that the linear cell array is excited and data acquired sequentially. Alternatively, the excitation/data acquisition scheme can involve simultaneous excitation and data acquisition from a plurality of pairs of opposing excitation cells and detector cells. Activation of cells can be accomplished through a number of multiplexers which are connected to each cell on the scanner plates. A multiplexer is an electronic chip which consists of one output and multiple input pins. The input pins are connected or disconnected from the output pin via internal electronic switches (relays). Multiplexers are significantly faster and produce less noise in comparison with electromechanical or mechanical switches. A single pass of the sequential or simultaneous excitation/data acquisition yields a projection corresponding to one angle. The scanner can be rotated incrementally to acquire sufficient data projections necessary for image reconstruction. Rotation of the scanner is optional and may depend on the type of imaging. Rotation is typically used for 3D imaging such as tomography. 2D imaging such as mammography may be achieved without rotation.

In silico phantom studies were conducted using an alternative parallel plate impedance scanner configuration comprising two parallel brass plates, each plate comprising a diaphragm, which can be opened and shut, on each plate side, the diaphragms maintained in opposing alignment. The opposing diaphragms are a functional equivalent of the opposing excitation cell and detector cell pairing. Movement of the diaphragms is coordinated so that the diaphragms are always maintained in opposing alignment and are synchronized to either be both open or both closed. In order to achieve an approximately linear relationship necessary for efficient EC reconstruction, a two-stage measurement scheme is executed. At each position along the diaphragm's motion direction, two capacitance measurements are conducted in sequence while the diaphragm is shut and then open. This pair of measurements is repeated at pixel size intervals until an object's field of view (FOV) is swept. EP of each pixel can be obtained easily using the corresponding EC value of the pixel and Equation 2.

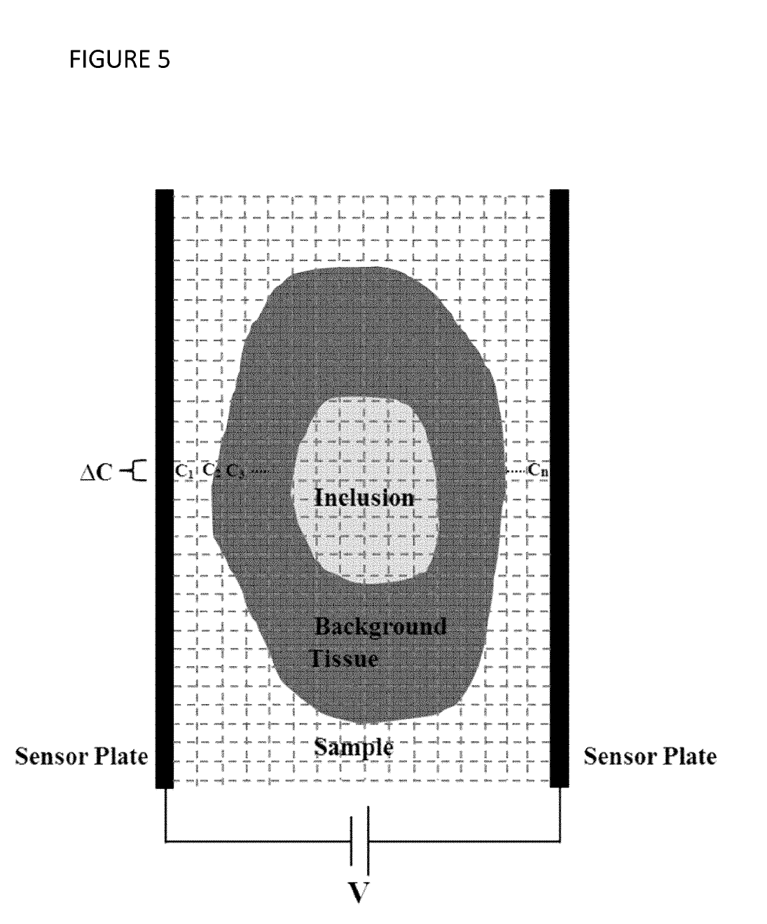

Discretization and EC Image Reconstruction with the diaphragm variant impedance scanner: FIG. 5 shows a schematic of two different tissue mimicking materials (e.g. background tissue and tumor) placed inside a parallel plate impedance scanner. The medium is discretized into small pixels with the size of the diaphragm hole using the shown grid. A medium column bridging the diaphragm holes consisting of pixel array labelled by C1, C2, C3, . . . , Cn is also shown. These C parameters represent the capacitance of material portion enclosed by a pixel which can be considered as a small capacitor. If the dimension of each pixel between the scanner's plates is assumed to be small enough, the permittivity and electric field within each pixel can be considered to be uniform while its direction is along the column's axis. Therefore, for each pixel Equation 1 can be approximated as follows:

C=εA/L (2)

where C, ε, A and L are the pixel's capacitance, permittivity, surface area and size, respectively.

Given the approximately 1D uniform electric field directed perpendicular to the plates' plane, pixels along each column can be approximated as series capacitors. As such, the relationship between the measured ΔC (i.e. capacitance difference between closed and open diaphragm states) and these elements is:

$$\frac{1}{Δ C} = \frac{1}{{C}_{1}} + \frac{1}{{C}_{2}} + … + \frac{1}{{C}_{n}} = \frac{\text{?}}{\text{?}} + \frac{{L}_{2}}{\text{?} {A}_{2}} + … + \frac{{L}_{n}}{\text{?} {A}_{n}}$$

$$\text{?} \text{indicates text missing or illegible when filed}$$

Assuming a uniform grid, this relationship can be simplified to the following:

$$\frac{1}{Δ C} = \frac{L}{A} {\sum }_{i = 1}^{n} \frac{1}{{ɛ}_{i}} \qquad ( 3 )$$

This is a linear relationship between the reciprocals of the measured data and tissue permittivity. In principle, the plates can be rotated around the object to acquire data pertaining to a number of projections sufficient for image reconstruction using linear back projection.

In silico Phantom Study for Linearity Assessment with Different Frequencies: to assess the effect of voltage source frequency in the diaphragm variant imaging system and determine the range of frequencies where the linear relationship given in Equation 3 is still valid, an in silico phantom study was carried out on two sets of phantoms. The first set involved three phantoms consisting of 60 mm×100 mm×60 mm block simulating background tissue with 10 mm, 15 mm and 25 mm diameter spherical inclusions, respectively. To mimic soft tissue stiffening resulting from cancer (e.g. breast cancer), the permittivity of inclusions for each frequency was assumed to be 3 times larger than the permittivity of the background tissue. The second set involved three phantoms consisting of 60 mm×100 mm×60 mm block simulating background tissue with 15 mm, 20 mm and 25 mm diameter spherical inclusions, respectively. In this set of phantoms, the permittivity of inclusion for each frequency was assumed to be 20 times lower than the permittivity of the background tissue to mimic bone inside muscle tissue. Each of these phantoms was assumed to be placed between the two parallel plates of the scanner such that the two diaphragms were aligned with the inclusion's centre during data acquisition. A square-shaped excitation voltage with 5 v amplitude and frequencies varying from 10 kHz to 10 GHz was applied to the scanner. A finite-element mesh consisting of ˜2.2 million 8-noded hexahedral elements was used for discretizing each phantom. The phantoms were analyzed under varying frequencies and corresponding electric fields were calculated using CST Studio Suite (Computer Simulation Technology AG, Darmstadt, Germany). Using this solver ΔC between the two diaphragm points arising from shutting and opening the diaphragm were also calculated and compared to the corresponding value obtained from Equation 3.

In silico Phantom Study for Linearity Assessment with Different Diaphragm Locations: to assess the validity of the linear approximation presented in Equation 3 along the plates' long axis (X direction), an in silico breast phantom study involving three phantoms was carried out. Each phantom consists of a 60 mm×100 mm×10 mm block simulating background mimicking healthy fibroglandular tissue. To evaluate inclusion size in this study, cylindrical inclusions of 15 mm, 20 mm and 25 mm in diameter were included in the phantoms to mimic breast tumors. The permittivity of inclusion for each phantom was assumed to be 3 times larger than the permittivity of the background tissue. Each of these phantoms was assumed to be placed between the two parallel plates of the scanner and the two diaphragms were moved along the X axis from −30 mm to 30 mm with 3 mm increments during data acquisition. The scanner's diaphragms' diameter was assumed to be 2 mm. A square-shaped excitation voltage with 5 v amplitude and 32 KHz frequency was applied to the scanner. In each step along the X axis, the capacitance of the scanner in the model with open and closed diaphragms was measured, and the deviation from Equation 3 linear approximation was calculated. Each phantom was discretized using ˜2.2 million 8-noded hexahedral elements to obtain its respective FE model which was solved using CST Studio Suite (Computer Simulation Technology AG, Darmstadt, Germany) to obtain ΔC at each diaphragm location. These values were compared to values obtained from Equation 3.

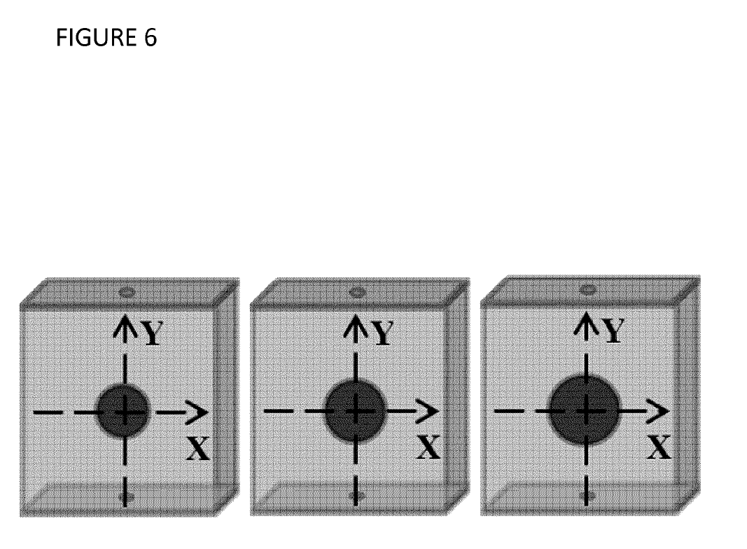

Image Reconstruction of a Phantom Using in silico Data: an in silico phantom study was conducted to investigate the quality of reconstructed permittivity images expected from the diaphragm variant impedance scanner in conjunction with the linear back projection algorithm. In this study three thin block 60 mm×60 mm×20 mm phantoms with round inclusions of 15 mm, 20 mm and 25 mm in diameter were used as illustrated in FIG. 6. The phantom was assumed to consist of tissues with permittivity values of 858 F/m and 2574 F/m for the background and inclusion, respectively. In order to generate capacitance data required for the permittivity image reconstruction, each phantom was discretized using 8-noded hexahedral elements. To ensure high accuracy, a fine mesh consisting of 1.2 million elements was used for modelling. Using CST

Studio Suite (Computer Simulation Technology AG, Darmstadt, Germany), the phantom and impedance scanner were modeled and the electric field resulting from an excitation voltage source with amplitude of 5 v and 32 kHz frequency was calculated. Using the obtained electric field in conjunction with the permittivity distribution, the scanner's capacitance was calculated. This calculation was performed with open and closed diaphragms with varying position ranging from −30 mm×30 mm along the plates. To obtain sufficient data necessary for image reconstruction using parallel beam projection algorithm, capacitance data were similarly obtained after rotating the two plates and once again varying the diaphragms position along the plates from −30 mm to 30 mm. This was performed along angles ranging from 0 to 180 degrees with 5 degree increments. Data obtained from this simulation was fed into a Linear Back Projection image reconstruction algorithm and a tomography permittivity image was reconstructed for each phantom.

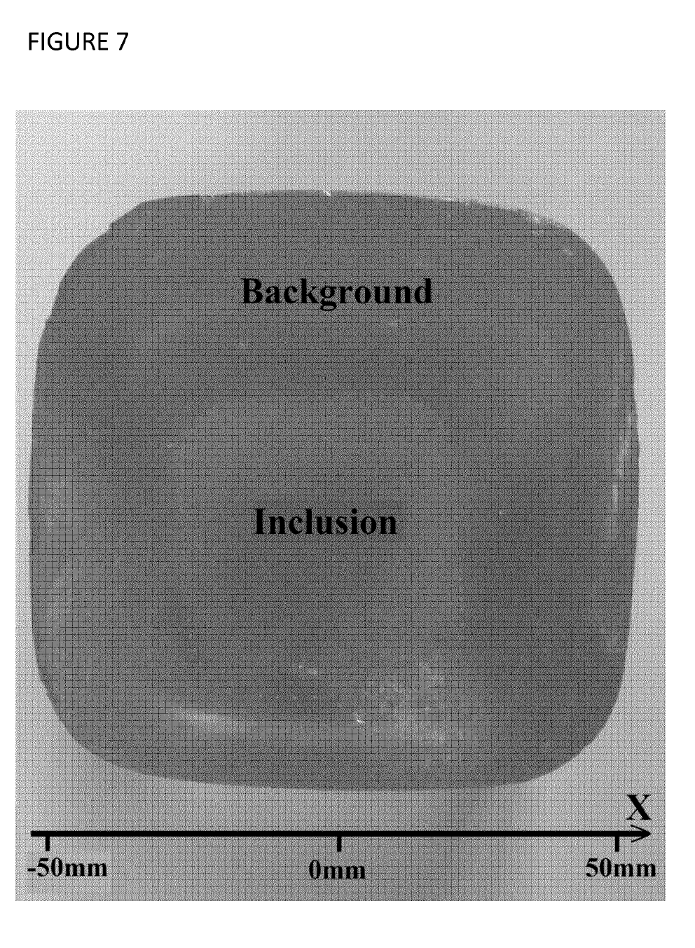

Tissue Mimicking Phantom Study: a study involving the tissue mimicking phantom shown in FIG. 7 was conducted. This phantom consists of a background and an inclusion constructed from gelatin, agar and salt. Dimensions of the background and inclusion are 100 mm×100 mm×90 mm and 50 mm×50 mm×25 mm, respectively. Permittivity values of the background and inclusion tissues were measured at 180 F/m and 420 F/m at 32 KHz, respectively. Each of these values was obtained by placing a small block shape representative sample of the material inside the impedance scanner and measuring the resultant capacitance value. Each permittivity value was then calculated using a 1-D minimization algorithm where the sample's finite-element model was used to calculate resultant capacitance corresponding to given permittivity value. This algorithm alters the permittivity of the sample's FE model systematically until the calculated capacitance matches the experimentally measured counterpart sufficiently closely. To construct the phantom, gelatin, agar and salt with various concentrations were used. For the background, 15% concentration of gelatin in distilled water was used while for the inclusion construction 15% gelatin and 1% agar in addition to 3% salt were used. The experimental setup consists of a data acquisition system with capability of measuring capacitance values as low as 10⁻¹⁸ F. The diameter of the diaphragms was 1.5 mm. The diaphragms on the scanner's plates were moved along X-axis from −50 mm to +50 mm with 5 mm increments. The data acquisition system was connected to the scanner's plates and continuously measured the scanner's capacitance at 32 KHz with open and closed diaphragms along this motion range. The excitation voltage of the scanner was set to 5V.

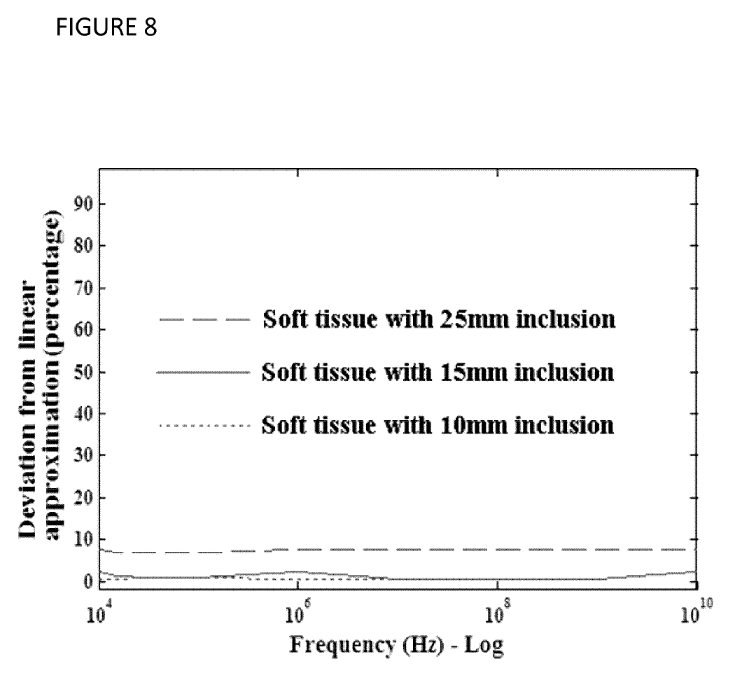

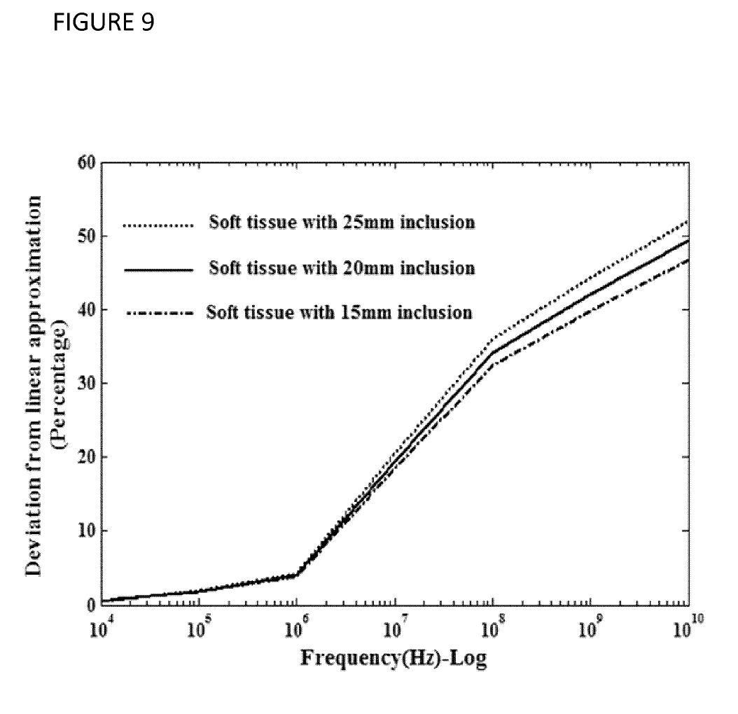

Results of in silico Phantom Study for Linearity Assessment with Different Frequencies: simulation results of the phantom study for frequency dependence assessment are illustrated in FIGS. 8 and 9. FIGS. 8 and 9 summarize the percentage error between theoretical ΔC obtained from CST studio and corresponding values obtained from Equation 3 for various frequencies. For all of the phantoms, at low frequencies (e.g. 100 KHz or lower) the electrical behavior of the impedance scanner becomes very close to linear. The maximum error occurs for the phantom with the 25 diameter inclusion. In this case, the maximum error with the inclusion with higher permittivity is ˜7% as shown in FIG. 8. This error is only ˜0.5% for the phantom where the inclusion has significantly lower permittivity in comparison to the background tissue as shown in FIG. 9 at frequencies lower than 100 KHz. This implies that at low frequencies, the electrical behaviour of the impedance scanner is such that the discretization where the tissue enclosed in columns bridging the two diaphragm points is approximated by series capacitors with a capacitance value of Cᵢ=εᵢ Aᵢ/Lᵢ, (see Equation 2) is a reasonably good approximation.

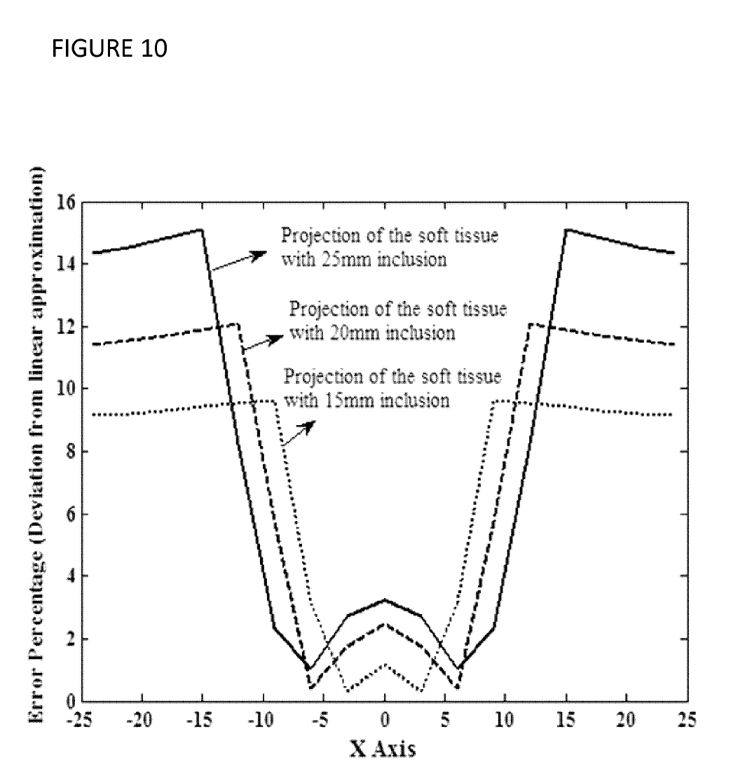

Results of in silico Phantom Study for Linearity Assessment with Different Diaphragm Locations: simulation results of a phantom study for diaphragm location assessment along the longitudinal axis (diaphragm's motion axis) of the scanner plates is illustrated in FIG. 10. FIG. 10 shows ΔC errors corresponding to deviation of the linear model from the numerical FE model of the phantoms used for linearity assessment with different diaphragm locations. These errors were obtained from simulation with an excitation voltage of 5 v amplitude and 32 kHz frequency with various diaphragm locations along the X axis. This figure shows that the errors increase sharply while approaching the inclusions' periphery and it remains almost constant outside the inclusions' width. As expected, the maximum errors correspond to the largest inclusion of 25 mm where the maximum errors within the inclusion and near its periphery are 3.7% and 14.8%, respectively.

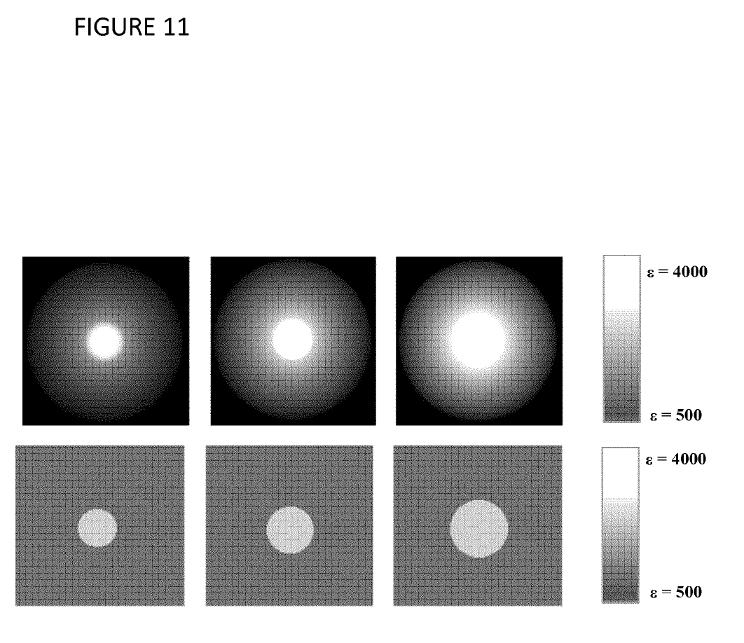

Results of Image Reconstruction of a Phantom Using in silico Data: FIG. 11 shows reconstructed permittivity images of the three tissue mimicking phantoms shown in FIG. 6. These images indicate that an artifact known as smoothing (blurring) effect are present around the inclusions in the reconstructed images. In order to mitigate this problem and reduce the smoothing effect, the images were segmented using thresholding technique. For this purpose different permittivity threshold values ranging from 2000 F/m to 2800 F/m were chosen to assess the sensitivity of resulting inclusion size with the threshold value. Segmented images obtained with threshold value of 2000 F/m are illustrated in the bottom row of FIG. 11. Segmentation results with the different threshold values indicate that the size of inclusions change by up to 5%, implying that the accuracy of inclusion geometry obtained by segmentation is not very sensitive to the threshold's value.

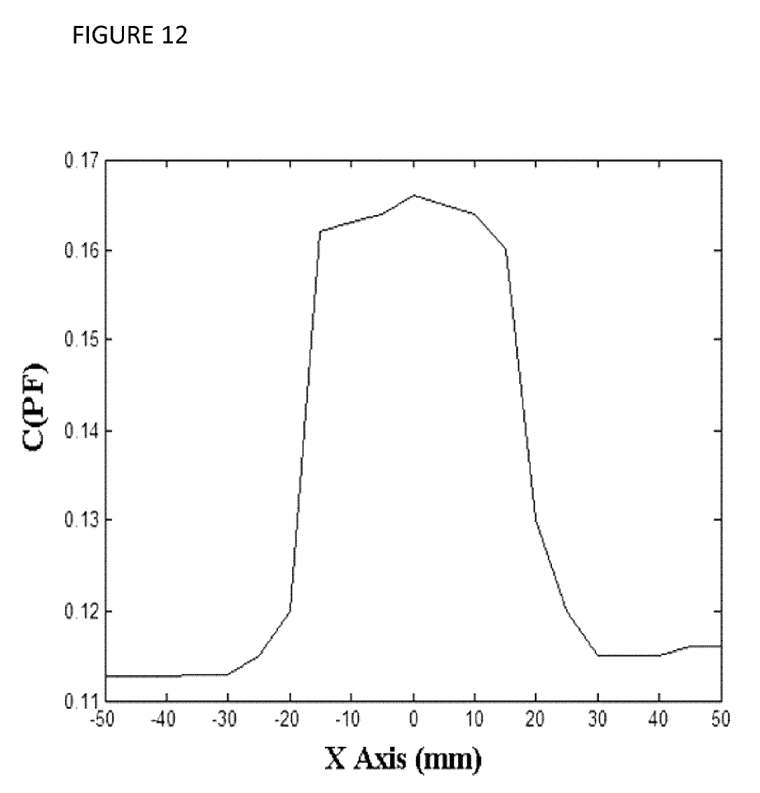

Results of Tissue Mimicking Phantom Study: FIG. 12 illustrates the acquired capacitance projection along the X axis. The amplitude of projection graph significantly rises as it reaches the inclusion and falls back to its initial value as it passes the inclusion which implies that the experimental setup was able to accurately detect the inclusion.

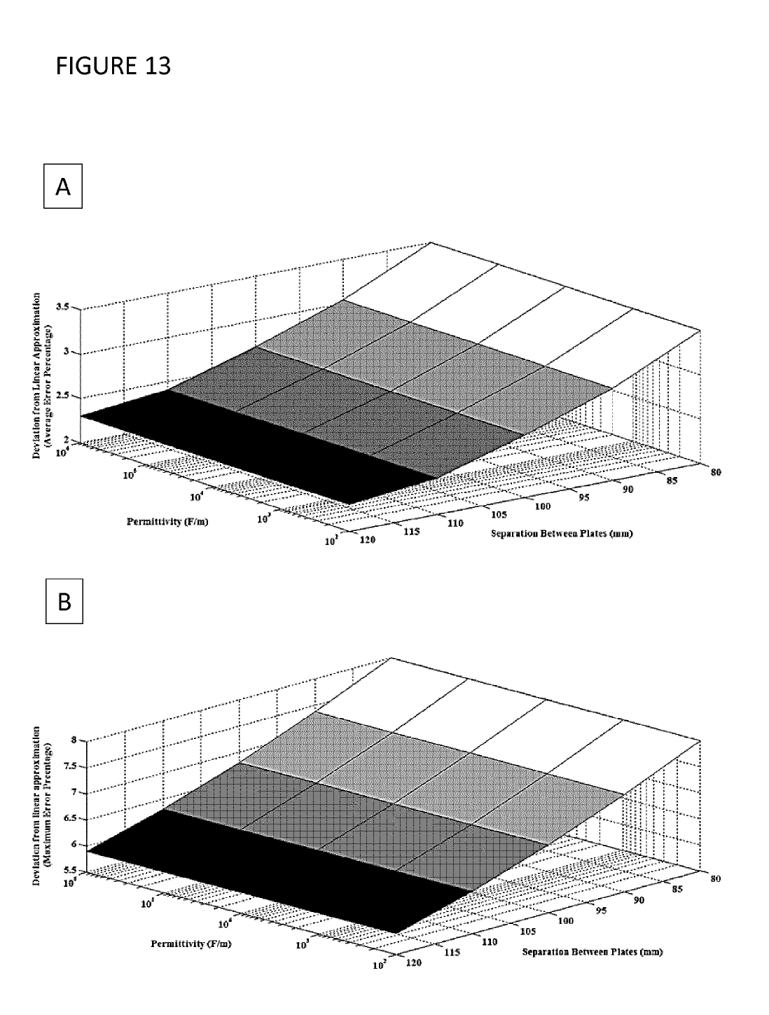

Linearity Deviation Metric with Simultaneous Firing of Cells of the Impedance Scanner variant shown in FIG. 1: an in silico phantom study involving a block shaped phantom with a 10 mm inclusion was conducted to assess deviation from the 1D linearity assumption as a function of permittivity and plate separation. Permittivity values ranging from 10² F/m to 10⁶ F/m consistent with the range of biological tissue permittivity were used for the background while 3 times greater permittivity values were used for the inclusion. Note that plate separation represents the breast's thickness after being held between the two plates of the impedance scanner. This parameter was varied between 80 mm to 120 mm. Deviation from the 1D linearity assumption was characterized using the metric Δc=100*|(C_{FEM}−C_{L}/C_{FEM}| where C_{FEM} and C_{L} are the capacitance between a cell pair using the FEM method taken as ground truth and using the analytical formula used to calculate capacitance of capacitors connected in series, respectively. Average and maximum values of this deviation metric are shown in FIGS. 13A and 13B, respectively, as functions of tissue permittivity and plate separation. FIGS. 13A and 13B indicate that there is very little variation of the deviation metric with respect to permittivity values for biological tissues while the maximum deviation from uniform 1D electric field is only 8%.

In a second set of experimental examples the impedance scanner is used to determine phase angle of impedance measurements of a sample held between the two parallel plates and process the phase angle data to generate an image of the sample.

Electrical impedance (EI) imaging modalities can address shortcomings of other medical imaging modalities currently used in medical imaging including, for example, cancer screening/imaging applications, such as X-ray, CT, ultrasound or MRI techniques. EI modalities use low energy electric field to probe and characterize electrical impedance of biological tissues. The use of non-ionizing electric field as well as the simplicity and low cost of these imaging modalities make them ideal for tumour screening/imaging including, for example, breast cancer screening. With regard to breast cancer screening/imaging EI modalities can include Electrical impedance tomography (EIT) and electrical impedance mammography (EIM). EIT and EIM produce images that display the distribution of tissue electrical impedance (electrical conductivity and electrical permittivity). Studies aimed at characterizing the electrical properties of normal and pathological tissue have shown that electrical conductivity and electrical permittivity of breast malignancies are significantly higher than those of benign and normal breast tissues

Despite recognized advantages of EI imaging, only a few studies have used EIM for breast cancer detection. Among them, Assenheimer et al. (Michel Assenheimer, Orah Laver-Moskovitz, Dov Malonek, David Manor, Udi Nahaliel, Ron Nitzan, Abraham Saad, The T-SCAN™ technology: electrical impedance as a diagnostic tool for breast cancer detection, Physiol. Meas., Vol. 22(1), February 2001, 1-8) demonstrated that current EIM technologies such as TransScan 2000 (Siemens Medical, Germany, and TransScan, Ramsey, N.J., USA), are only capable of detecting high impedance inclusions located close to the breast surface. This research introduces a novel EIM technique which uses an electrical impedance imaging system comprising a parallel plate scanner. This investigation involves in silico and tissue mimicking phantom studies conducted to demonstrate its application for medical diagnosis including, for example, breast cancer screening. A description of the TransScan device may be found, for example, in U.S. Pat. No. 6,560,480 issued 6 May 2003.

The electromagnetic field generated by applying current density to a body surface is governed by Maxwell's equations. For a nonmagnetic material such as biological tissues, the general form of Maxwell's equations in the time domain with the inclusion of displacement current and continuity equation is as follows:

$$\begin{matrix}\frac{\partial ρ ( r , t )}{\partial t} + ∇ \cdot J ( r , t ) = σ & ( 4 ) \\ ∇ \cdot D ( r , t ) = ρ ( r , t ) & ( 5 ) \\ ∇ \times H ( r , t ) = J ( r , t ) + \frac{\partial D ( r , t )}{\partial t} = σ E ( r , t ) + {J}_{e} ( r , t ) + \frac{\partial D ( r , t )}{\partial t} & ( 6 ) \\ ∇ \cdot B ( r , t ) = 0 & ( 7 ) \\ ∇ \times E ( r , t ) = - \frac{\partial B ( r , t )}{\partial t} & ( 8 )\end{matrix}$$

where ρ(r,t) is the electric charge density, J is the electric current density, E is the electric field, D=εE is the electric displacement current, ε is the electric permittivity, B is the magnetic field, H=B/μ is the magnetic intensity and μ is the magnetic permeability which is considered to be the same as the permeability of vacuum for biological tissues. In this study, the external magnetic field is assumed to be negligible (B=0). A further assumption is that impedance measurement is performed at low frequencies (1 MHz or lower) where the frequency of the voltage source is low enough for the EM propagation delay to be neglected. Using the phasor format of Equations 4 to 8 and dropping the time harmonic, leads to the following equations in the frequency domain. This was performed to facilitate the equations' computational solution consistent with the COMSOL Multiphysics software package (COMSOL, Inc., MA, USA) used in this second set of experiments.

∇·J(r,ω)=Qⱼ(r,ω) (9)

J(r,ω)=σE(r,ω)+jωD(r,ω)+Jₑ(r,ω) (10)

E(r,ω)=−∇V(r,ω) (11)

where Qⱼ represents current source, σ is tissue electrical conductivity, ω is the natural frequency, Jₑ is an externally induced current density and V is the electric potential. COMSOL finite element method (FEM) can be used to solve Equations 9 to 11 to obtain the impedance amplitude and phase angle in the breast models involved in this second set of experiments.

Similar to x-ray mammography where the breast is placed in a parallel-plate compression unit and projections of x-ray are measured and converted into mammograms, in the EIM technique for this second set of experiments, the breast is gently compressed between the two parallel plates of an impedance scanner. While the breast is gently compressed, the electrical impedance its tissue is measured as projection data before they are converted into a mammogram. Depending on the excitation frequency in the proposed technique, different types of image reconstruction methods such as image impedance, resistance, capacitance and phase angle may be employed to generate respective images. While imaging impedance and resistance are feasible at all excitation frequencies, for the capacitance and phase angle imaging, choosing a suitable range of excitation frequency can be significant. This dependence on choosing a suitable range of excitation frequency is such that at some frequencies (e.g. higher than 30 kHz), the phase angle of the measured impedance becomes so small (close to zero) that the capacitance, permittivity and phase angle image reconstructions are not feasible (capacitance and permittivity cannot be determined when the phase angle is zero). Depending on the anatomical site or tissue sample, a frequency cut off or an upper frequency limit (for example, 10 kHz, 5 kHz or even lower) can be defined where the excitation frequency is less than the cut-off/upper limit. The frequency cut-off or upper limit may be adjusted for different tissues or anatomical sites and beyond such a frequency, C, EC and phase angle imaging may not be feasible.

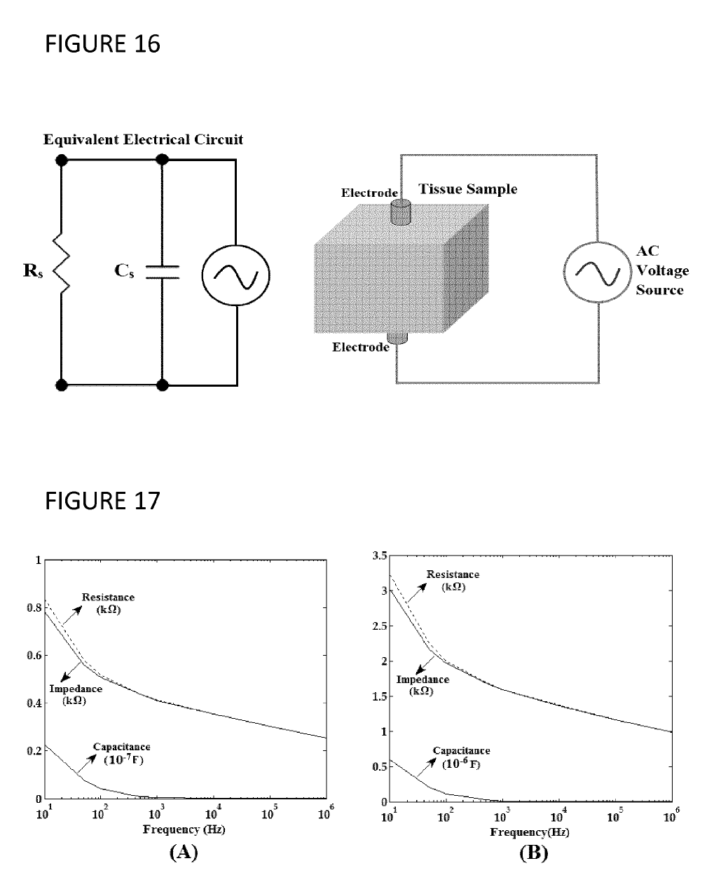

In order to study the electrical behaviour of a biological tissue, a proper electrical model is useful. A lumped electric model (equivalent circuit) of a tissue part of the breast located between two electrodes of the two parallel plates at low frequencies is shown in FIG. 16. It consists of a parallel resistor and capacitor.

It is noteworthy that this electrical model of biological tissues, which is used extensively in the literature, has an additional series resistor (not shown) with capacitance Cₛ. However, at low frequencies, the value of this resistor, which represents the resistance of intracellular fluids, becomes negligible. The relationship between electrical impedance, resistance, capacitance, and phase angle of a biological tissue sample derived from its equivalent circuit, is:

Zₛ∠θₛ=[(Rₛ/ωCₛ)/(Rₛ²+(1/ωCₛ)²)^{1/2}]∠+90°+Arctg(1/RₛCₛω) (12)

where Zₛ and θₛ are the measured amplitude and phase angle of the tissue's electrical impedance, ω is the natural frequency of the excitation signal, and Rₛ and Cₛ are the tissue's electrical resistance and capacitance, respectively.

In order to examine how the impedance components of a typical biological tissue (e.g. adipose) changes with frequency, a computational simulation was performed involving an adipose tissue specimen. An electrical model of a 50 mm×50 mm×50 mm block-shaped adipose tissue specimen was constructed, and its electrical impedance (Zs ∠θs) was measured at frequencies of 10 Hz to 1 MHz via simulation using COMSOL. The electrical conductivity and permittivity of the tissue specimen at these frequencies, which were input to reconstruct the model, were obtained from the literature (C Gabriel, S Gabriel and E Corthout, The dielectric properties of biological tissues: I. Literature survey, Phys. Med. Biol. 41 (1996) 2231-2249; S Gabriel, R W Lau and C Gabriel, The dielectric properties of biological tissues: II. Measurements in the frequency range 10 Hz to 20 GHz, Phys. Med. Biol. 41 (1996) 2251-2269). The measurement was conducted using two different configurations, leading to two corresponding finite element (FE) models. In one configuration the specimen was assumed to be placed between two cylindrical brass electrodes with a radius of 1.5 mm and height of 2 mm. In the other configuration, the specimen was assumed to be held between the parallel plates of an imaging scanner that is a variant of the scanner shown in FIG. 1 devoid of an insulation layer and having guards that have a surface area equal to the surface area of their respective corresponding cells. Each of these models consisted of ˜2.2 tetrahedral finite elements.

Using COMSOL solver in conjunction with Equation 12, the capacitance and resistance data of the adipose tissue specimen at the 10 Hz-1 MHz frequency range were obtained for each configuration. These data, which are illustrated in FIG. 17, show that at frequencies higher than 1 kHz, the adipose tissue capacitance component diminishes, hence the tissue's impedance becomes predominantly resistive at such frequencies. This implies that the reconstruction of capacitance, permittivity and phase angle images that involve the capacitive component of the tissue's impedance are advantageously generated at excitation frequencies lower than 1 kHz. Based on these observations, the following three types of image reconstruction can be derived.

First type of image reconstruction: Electrical Resistivity and Conductivity Image Reconstructions in EIT and EIM. Electrical conductivity image reconstruction is the easiest and most common type of electrical impedance image reconstruction. This method has been used in the majority of EIT (electrical impedance tomography) applications in the past three decades. The following equation shows the fundamental relationship between tissue electrical resistivity and its conductivity,

$$R = \frac{V}{I} = \frac{\int E \cdot dl}{∮ σ E \cdot ds} \qquad ( 13 )$$

where R is the tissue electrical resistance, V is the potential difference between the two electrodes where the voltage is being measured, I is the electric current, E is the electric field and a is the tissue electrical conductivity. In the context of breast imaging, electrical resistance and electrical conductivity image reconstruction may be performed in the whole frequency range of 10 Hz-1 MHz, as according to FIG. 17 the measured resistance at this frequency range is appreciably high. As such, in the majority of EIT image reconstruction methods which mainly use frequencies higher than 1 kHz, the measured amplitude of tissue's impedance is simply approximated by its electrical resistance. However, the major problem with conductivity image reconstruction stems from the complex relationship between R and σ and its high sensitivity to the electric field. Consequently, this type of image reconstruction leads to an ill-posed problem, which requires iterative and non-linear image reconstruction algorithms. Furthermore, previous studies have shown that the variation range of conductivities for biological tissues at 10 Hz-20 GHz is limited. This implies that conductivity and resistance imaging of biological tissues may not produce images with high contrast.

The following equation, which is derived from the lumped electrical model of the tissue (parallel capacitor and resistor in FIG. 16), shows the relationship between the tissue resistance (Rs), their electrical impedance (Zs) and phase angle (θs).

$${R}_{s} = \frac{{Z}_{3} tg ( 90 + {θ}_{3} )}{\sqrt{( 1 + {tg}^{2} ( 90 + {θ}_{3} )}} \qquad ( 14 )$$

In EIM, resistance image reconstruction involves obtaining resistance projection data for each point on the breast surface plane, and converting this data into 2D mammograms. As such, the breast tissue's impedance projections on the breast surface plane was measured using a variant of the scanner shown in FIG. 1—a scanner devoid of an insulation layer 22 and having guards that have a surface area equal to the surface area of their respective corresponding cells. Then by using Equation (14), the resistance projection data of the breast tissue was calculated and converted into 2D resistance mammograms. As solving Equation 13 for σ is not convenient, to obtain an estimate of the breast tissue's conductivity projection on the breast surface plane, an assumption of uniform electric field leading to the inverted resistance image can be used.

Second type of image reconstruction: Electrical Permittivity and Capacitance Image Reconstructions. Electrical permittivity is an intrinsic property of materials, which may be obtained via the material's electrical capacitance. For measuring tissue electrical capacitance, the amplitude and phase angle of the tissue's impedance must be measured. The following equation shows the relationship between the tissue capacitance (Cs), their electrical impedance (Zs) and phase angle (θs) based on the lumped electrical model shown in FIG. 16.

$${C}_{s} = \frac{1}{{Z}_{3} ω \sqrt{( 1 + {tg}^{2} ( 90 + {θ}_{3} )}} \qquad ( 15 )$$

According to FIG. 17, for a breast adipose tissue specimen placed between two electrodes, measuring the capacitance (Cs) and phase angle (θs) at frequencies higher than 1 kHz may not be feasible, as the tissue capacitance becomes too small to be reliably measured. As such, for breast imaging, capacitance, permittivity and phase angle image reconstructions performed at high frequencies (eg., greater than 5 KHz) are of reduced reliability. However, at lower frequencies (e.g. 1 KHz or lower) where the electrical capacitance is sufficiently large, a reliable measurement of Cs is feasible.

Measurement of tissue electrical permittivity (ε) can be achieved by measuring its electrical capacitance (Cₛ) as direct measurement of permittivity is not feasible. The following equation shows the fundamental relationship between electrical capacitance (C) and electrical permittivity (ε):

$$C = \frac{Q}{V} = \frac{∮ \text{?} E \cdot ds}{\int E \cdot dl} \\ \text{?} \text{indicates text missing or illegible when filed} \qquad ( 16 )$$

where Q represents the electric charge, V is the potential difference between the two electrodes where the measurement is performed, E is the electric field and ε is the tissue electrical permittivity. This equation shows that the relationship between C and ε is complex and highly dependent on the electric field. As such, tissue permittivity image reconstruction may also lead to ill-posed problems that require iterative and non-linear inverse problem solution algorithms. However, as the variation range of permittivity of biological tissues is very broad in comparison with that of conductivity, permittivity and capacitance imaging is expected to produce images with higher contrast; hence they are preferable over resistance and conductivity imaging.

In EIM, capacitance image reconstruction involves obtaining capacitance projection data for each point on the breast surface plane followed by converting the data into 2D capacitance mammograms. In this study we measured the capacitance projections of the breast models on their surface plane using a variant of the scanner shown in FIG. 1—ie., devoid of an insulation layer and having guards that have a surface area equal to the surface area of their respective corresponding cells. Using Equation 15, the capacitance projection data of the breast tissue was calculated from the impedance data before they were converted into 2D capacitance mammograms. As solving Equation 16 for ε is not feasible, to obtain an estimate of the breast tissue's permittivity projection on the breast surface plane, the capacitance image can be used as capacitance and permittivity are approximately proportional.

Third type of image reconstruction: Phase Angle Image Reconstruction. Impedance phase angle of a tissue (θs) may be obtained from Equation 12, leading to the following equation:

θₛ=−90°+Arctg(1/RₛCₛω) (17)

Using the discrete form of Equations 10 and 13 leads to:

$$\frac{1}{{R}_{S} {C}_{S} ω} = \frac{{\sum }_{i = 1}^{m} {σ}_{i} {E}_{i} \cdot Δ {S}_{i}}{ω {\sum }_{i = 1}^{m} {E}_{i} \cdot Δ {L}_{i}} \times \frac{{\sum }_{i = 1}^{m} {E}_{i} \cdot Δ {L}_{i}}{{\sum }_{i = 1}^{m} \text{?} {E}_{i} \cdot Δ {S}_{i}} \\ \text{?} \text{indicates text missing or illegible when filed} \qquad ( 18 )$$

Assuming equal ΔSᵢ and ΔLᵢ spacing within each element where tissue homogeneity is a good approximation, this relationship may be simplified to the following:

$$\frac{1}{{R}_{S} {C}_{S} ω} = \frac{σ}{ɛω} \qquad ( 19 )$$

Substituting the above in Equation 18 leads to:

$${θ}_{s} = - 90 ° + Arctg ( \frac{σ}{ɛω} ) \qquad ( 20 )$$

This equation shows that, unlike resistance and capacitance that depend on the electric field and element geometry in addition to the tissue intrinsic properties, the impedance phase angle is dependent on the intrinsic electrical properties of the tissue only. As such, phase angle images are expected to be of higher quality compared to resistance and capacitance images. Moreover, phase angle imaging of the breast is feasible at lower frequencies (e.g. <1 kHz) only where the capacitance component of the measured impedance is non-zero.

Configuration of an Electrical Impedance Mammography System. An electrical impedance mammography scanner was constructed. It comprises two parallel plates where the breast is placed in between before image acquisition is performed. One plate is used for excitation while the other is a detector plate. The excitation plate includes the excitation board while the detector plate is a hand-held plate which can include a detector board and analog and digital boards. The excitation board comprises a large conductive plate and an electronic board on the back, which generates the excitation sinusoidal signals with selectable frequency at 5 Vp-p. The detector board consists of a 1-D circular cells array spaced at about 5 mm increments along the top surface of the breast to scan its entire volume. The 1-D array consists of thirty circular cells. The radius of each cell is 1.5 mm; each one is separated from the next by a gap of 0.125 mm on the printed circuit board (PCB). For data acquisition, the breast was squeezed gently between the detector plate and the excitation plate. The impedance signals, which were obtained from the cells of the 1-D array, were first amplified by the analog circuit board before they were sent to the digital circuit board. The digital circuit board consists of multiple 24 bit analog to digital converters (AD7766, Analog Devices, Massachusetts, USA) and a microcontroller (ATmega320, Atmel, California, USA). AD7766 converts the analog impedance signal into 24 bits digital packets and sends them through the USB port to a computer. A Matlab (MathWorks, Massachusetts, USA) code on the computer side, which is connected to the scanner (more specifically, the microcontroller) through the USB port, receives the digital impedance data and converts them into 2D digital images. The microcontrollers on the digital board of the scanner does all the coordination between the A/D converter and computer. The whole procedure can be completed in less than 10 seconds.

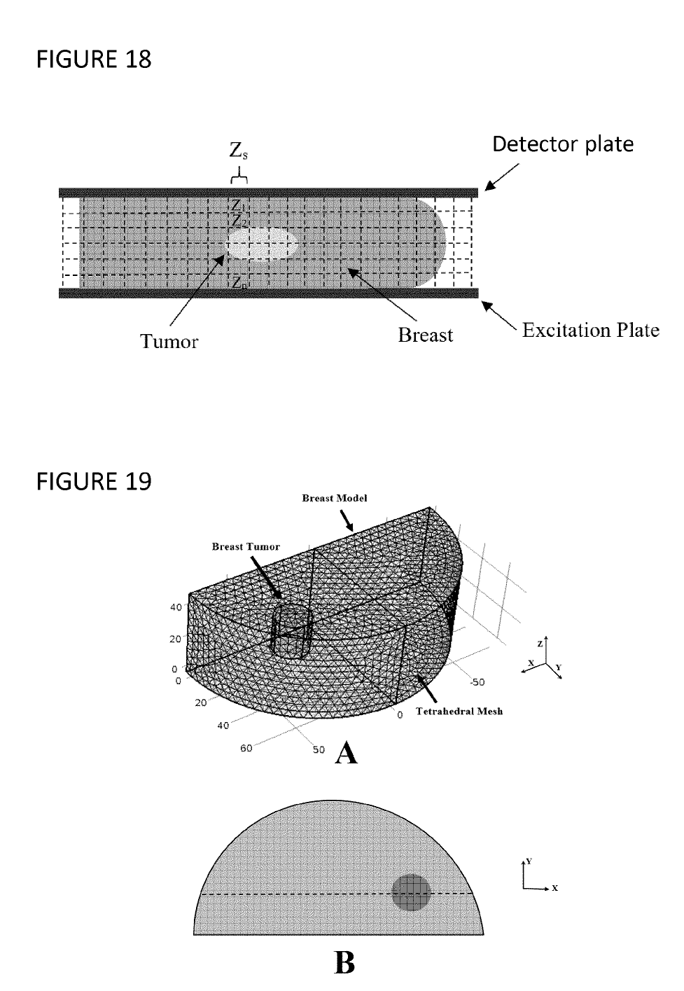

A schematic of the scanner in this second set of experiments is illustrated in FIG. 18. Each conductive cell on the detector board is connected to an impedance measurement circuit that measures the impedance magnitude and phase angle of the adjacent breast tissue with 0.1Ω and 0.01° accuracy, respectively.

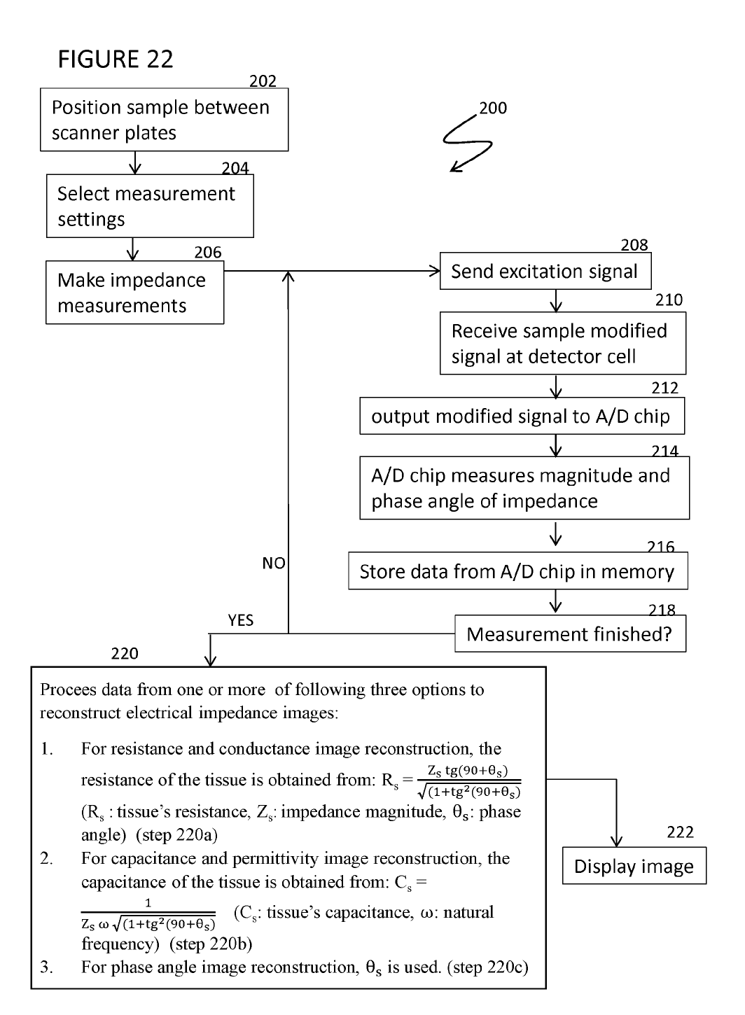

A schematic of the impedance imaging method 200 is shown in FIG. 22. The scanner is suitably positioned 202 so that the sample to be analyzed (for example, breast in the case of a mammogram) is placed between the plates of the impedance scanner and gently compressed. Any measurement settings such as frequency or applied voltage may be selected and set 204 depending on sample type or size as desired. Impedance measurements are then initiated 206. The impedance measurement starts with generation of an excitation signal 208 which is communicated to the excitation cell and emitted into the sample. The electric current that passes through the tissue sample is received at a detector cell 210, and the signal modified by the tissue is communicated to an analog to digital (A/D) converter electronic chip. The A/D chip, measures the magnitude and phase angle of the tissue modified analog signal (i.e. electric current) by comparing it with a reference signal and converts the measured analog quantities (magnitude and phase angle) into binary codes 216. The binary codes can be stored in a memory 216. Steps from generation of an excitation signal 208 to storage of data from the A/D chip in memory 216 can repeat through a plurality of cycles (for example, during sequential firing of excitation/detector cell pairs) until the impedance measurement is finished 218. The A/D chip data is then communicated to a computer/processor via a serial interface. The data can then be processed using an image reconstruction computer code 220 leading to display of the image 222. The image reconstruction computer code can be varied to accommodate different imaging techniques described herein including, for example, image reconstruction computer code based on resistance/conductivity 220a, capacitance/permittivity 220b, phase angle 220c, or any combination thereof.

The tissue's impedance components (the tissue's resistance and capacitance) measured by the scanner, can be described theoretically by Equations 13 and 16. As these equations show, the measured tissue's resistance (R) and capacitance (C) are highly dependant on the electric field (E) inside the tissue between the parallel plates, the contact area of each conductive cell (A), separation between the scanner plates (L) and dielectric property of the tissue (σ and ε). If the electric field between the scanner plates was uniform, the Equations 13 and 16 could be simplified to

$$R = \frac{L}{σ A} and C = \frac{zA}{L} ,$$

respectively. This implies if E was uniform, for a constant A and L, the measured tissue's resistance and capacitance would be functions of tissue dielectric properties, σ and ε only.

Methods of in silico Breast Phantom Experiment. To assess the capability of the scanner for breast cancer detection, and to evaluate the three types of image reconstruction (ie., conductance, permittivity and phase angle imaging) a series of computer simulations were carried out on a phantom following the configuration shown in FIG. 19. The simulations were carried out using the COMSOL Multiphysics software package. The phantom mimics a breast gently compressed by two plates, hence it consists of a half-cylinder with a radius of 75 mm and height of 50 mm. It embeds a cylindrical inclusion with a radius and thickness of 10 mm and 20 mm, respectively. In order to increase the simulation's realism, the inclusion was positioned as illustrated in FIG. 19 in order to mimic the upper outer quadrant where the majority of breast cancer tumors are found. The location of the inclusion along the height of the cylindrical phantom was set to be variable such that the inclusion's centre was located at the centres of the bottom, middle and top thirds along the height. The permittivity and conductivity values assigned to the breast model were chosen based on values reported in the literature for breast tissue at 0.5 kHz. The inclusion's permittivity and conductivity values were assumed to be 6 and 8 times higher than normal breast tissue's conductivity and permittivity, respectively. The breast phantom's FE mesh, which is illustrated in FIG. 18, consisted of ˜2.7 million tetrahedral elements. Similar to the scanner, one conductive plate was modeled to touch the breast model from the bottom to provide an excitation signal, while the top plate (detector) was considered to measure the impedance. The detector plate consisted of 30 circular conductive cells, each with a radius of 1.5 mm and separation of 0.2 mm. The COMSOL solver used the FEM approach to numerically solve Maxwell's equations and compute the amplitude and phase angle of the electric current that passed through each detector cell. From these computations, the impedance values of the breast tissue located between each detector cell and excitation cell was acquired. To obtain the projected mammography image of the resistance and capacitance, the projection value for each cell was calculated using Equations 14 and 15.

Methods for Tissue Mimicking Breast Phantom Experiment. A tissue mimicking phantom study was performed to assess the effectiveness of the three types of imaging techniques. A gelatine phantom was prepared following the general shape of the in silico phantom shown in FIG. 19, the gelatine phantom comprising a half-cylinder background tissue embedding a cylindrical inclusion constructed of gelatin and common salt. The background half cylinder part was 150 mm in diameter and 50 mm in height while the diameter and height of the cylindrical inclusion were both 20 mm. Along the phantom's height, the inclusion was placed in the middle.

The conductivity and permittivity of the background and inclusion tissues were measured independently prior to image data acquisition. At 0.5 kHz, their conductivity were 0.23 S/m and 1.2 S/m while their relative permittivity were 1,084,454 and 8,546,138 for the background and inclusion, respectively. Each of these values were obtained by placing a small block shape representative sample between the two electrodes of the apparatus shown in FIG. 16 followed by measuring the resultant resistance and capacitance values. The conductivity and permittivity values of each tissue were then calculated using a 2-D optimization algorithm where the sample's FE models were used to calculate the resultant resistance and capacitance values corresponding to the current estimates of conductivity and permittivity values in the optimization process. The algorithm altered the conductivity and permittivity of the sample's FE model systematically until the mismatch between the calculated and experimentally measured resistance and capacitance values was a minimum. To construct the phantom, gelatin and common salt with various concentrations were used. For the background, 12% concentration of gelatin in distilled water was used while for making the inclusion 12% of gelatin and 0.09% common salt was used. The experimental setup consisted of the data acquisition described above where an excitation voltage of the scanner was set to 5 Vp-p at 0.5 kHz.

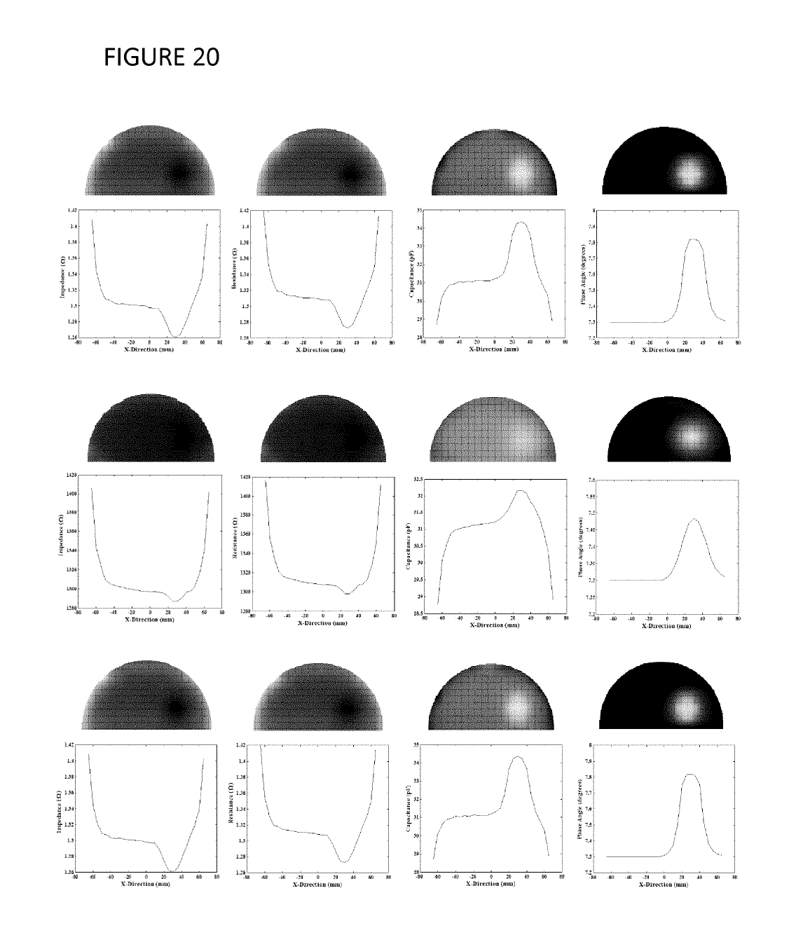

Results of in silico Breast Phantom Experiment. Images reconstructed from the in silico breast phantom are shown in FIG. 20. They show 2D mammography images obtained by projection of the impedance, resistance, capacitance, and phase angle. The impedance technique images are shown as a reference point for comparing the three image reconstruction techniques described above: resistance/conductivity, capacitance/permittivity, and phase angle. The impedance technique is based on measuring impedance amplitude/magnitude and produces images based on the projection data without applying an image reconstruction. The rows of images of FIG. 20 correspond to three different tumor positions along the height (z-axis) of the breast phantom. The images were produced from raw simulation data with no additional filtering or manipulation. As described above, the permittivity images are similar to the capacitance images while the conductivity images are similar to inverted resistance images. Thus, the permittivity and conductivity images of the breast phantom are not shown. Variation profiles of the measured impedance, resistance, capacitance and phase angle of the in silico breast phantom along the section crossing the inclusion (shown in FIG. 19B) are also illustrated in FIG. 20. Due to symmetry, the reconstructed images of the phantom with the inclusion located at the centres of bottom and top thirds along the height of the phantom (rows 1, 2 and 5, 6) are identical. As expected, image contrast pertaining to these bottom third and top third locations is higher compared to the case where the inclusion is located in the middle of the phantom's height. This is particularly more important with the impedance and resistance images where the respective images can hardly detect the inclusion. Among the reconstructed images, the capacitance and phase angle images exhibited higher contrast and better quality compared to the impedance and resistance images.

The results revealed that there are artifacts seen as intensity variations around the phantom and inclusion's periphery in the reconstructed impedance, resistance, and capacitance images. These artifacts were caused by the nonlinearity and non-uniformity of the electric field. This led to about 9% higher measured impedance and resistance, and about 10% lower measured capacitance around the peripheries as shown in the 2nd, 4th, and 6th rows of FIG. 20.

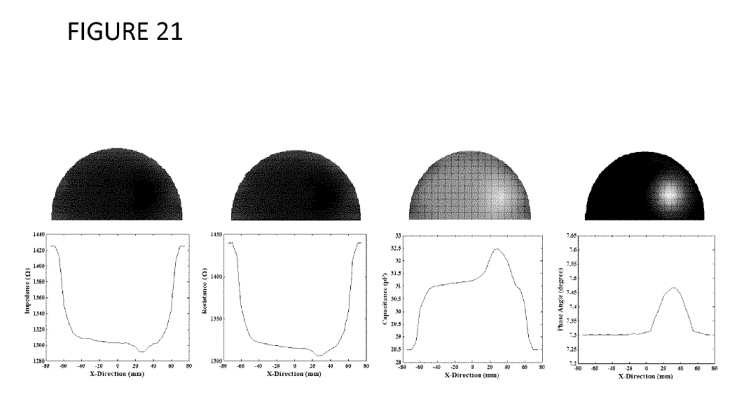

Results of Tissue Mimicking Breast Phantom Experiment. Reconstructed images obtained from the tissue mimicking breast phantom study are shown in FIG. 21. Pixel size in these images is 3 mm×5 mm. FIG. 21 demonstrates that the inclusion can be clearly distinguished from the background on the capacitance and phase angle images. Similar to the reconstructed images obtained from the in silico breast phantom study, the inclusion in the impedance and resistance images of the gelatin phantom cannot be clearly differentiated from its background. The second row of FIG. 21 illustrates the variation profiles of the impedance, resistance, capacitance, and phase angle signals along the section crossing the inclusion as shown in FIG. 19B.

The in silico and tissue mimicking phantom studies indicated that, among the various tested imaging techniques, the permittivity, capacitance and phase angle images were shown to be more effective than the impedance, resistance and conductivity images. Moreover, the studies demonstrated that the phase angle image reconstruction was capable of producing the highest quality images consistent with Equation 20, which implied strict dependence on the tissue intrinsic properties.

Experimental results described herein suggest that breast inclusions with higher dielectric values are highly detectable when they are located in the top outer quadrant of the breast. This may be highly advantageous for breast cancer detection, as previous research has shown that the majority of cancer tumors form in the top outer quadrant of breast. Higher conductivity and permittivity of an inclusion also leads to improved tumor detection characterized by higher image contrast. Results provided are conservative, as only conservative increases of 6 and 8 times higher values of conductivity and permittivity were assigned to the inclusion in the in silico and tissue mimicking phantom studies relative to background, compared to previous studies that have established the dielectric values of breast cancer tumors at 20-40-fold higher than those of normal breast tissue. Experimental results indicate that the projection images are able to properly capture the location of inclusions with higher dielectric parameter values. However, the size of the inclusion in these images increase with depth. For example, the inclusion in the image corresponding to the case where the inclusion is located in the breast's mid-height appears more diffused (3^{rd} row of FIG. 20), hence its sizes is overestimated in comparison with the images corresponding to cases where the inclusion is located in the top or bottom one-third heights. These size variations are due to the electric field non-uniformity. Results obtained from this investigation indicate that, among images produced by the various image reconstruction techniques, the phase angle image is superior in terms of cancer detectability. These results also suggest that the proposed EIM technique is capable of detecting inclusions located deep inside the breast while other EIM technologies such as TransScan are only capable of detecting inclusions located close to the breast surface.

The impedance scanner described herein provides several advantages over existing technologies. The scanner can measure capacitance as low as Femtofarad. Using in silico phantom studies, it was shown that at low frequencies, for example 1 Hz to 10 kHz the average error due to deviation from the linear equation approximation is reasonably low, especially at the centre of inclusion. As such, the scanner can readily operate at low frequencies, leading to reasonably good quality images constructed using linear back projection. FIG. 10, which was obtained from an in silico phantom study involving block shape phantoms with inclusions with various sizes, indicated that the maximum deviation from the linear equation approximation occurs at the periphery of the inclusions which suggests that the blurring artifact around the periphery of inclusions in the reconstructed images is caused by the mentioned approximation error. This artifact, which is also known as smoothing effect, is quite common in electrical properties imaging. Results indicate that the quality of images obtained by the impedance scanner described herein is comparable or superior to those of prior art ECT and EIT. Moreover, image reconstruction is carried out using straight forward linear back projection in contrast to nonlinear optimization techniques associated with prior art ECT and EIT techniques. As expected, a trade-off exists between the contrast and resolution of the impedance scanner imaging system. In other words resolution and contrast of the imaging system is determined by the size of diaphragms/cells such that smaller diaphragms/cells produce smaller and narrower perturbation in the scanner's electric field and can produce images with high resolution and small dynamic range while large diaphragms/cells produce larger perturbation with higher SNR and high image dynamic range but with lower resolution. It was concluded from the results of the in silico and tissue-mimicking phantom studies that inclusions with different dielectric properties (resistance, conductivity, permittivity, capacitance and phase angle) compared to their surrounding tissues are highly detectable using the image reconstruction techniques described above, particularly the permittivity, capacitance and phase angle techniques. Being safe and low-cost are two further advantages that the impedance scanner offers. Results are encouraging and indicate that the impedance scanner is capable of detecting tissue abnormalities effectively, rendering it a safe, effective and inexpensive tool for cancer screening applications.

Several illustrative variants have been described above. Further variants and modifications are described below. Moreover, guiding relationships for configuring variants and modifications are also described below. Still further variants and modifications are contemplated and will be recognized by the person of skill in the art. It is to be understood that guiding relationships and illustrative variants or modifications are provided for the purpose of enhancing the understanding of the person of skill in the art and are not intended as limiting statements.

Frequency of the impedance scanner electric field will typically range between 1 Hz and 1 MHz for medical applications. More specifically, for most medical imaging/screening applications the frequency will be less than 100 KHz and may be optimized for a specific tissue type. In many examples of medical imaging/screening applications the frequency will range between 10 Hz and 10 KHz. In examples of medical imaging/screening applications, including, for example, human breast tumors, the frequency will often be set in a range with a lower limit of 10 Hz and an upper limit that may be less than 5 KHz, less than 4 KHz, less 3 KHz, less than 2 KHz, less than 1 KHz or less than 0.5 KHz. In further examples of medical imaging/screening applications for human breast tumors, the frequency can be set in a range with a lower limit of about 100 Hz and an upper limit that may be less than 5 KHz, less than 4 KHz, less 3 KHz, less than 2 KHz, less than 1 KHz or less than 0.5 KHz.

Experiments indicate that both circular and non-circular (eg., square) shaped cells generate equally uniform 1D electric field. Thus, the shape of cells can be modified as desired. Electric field uniformity implies that the value and direction of the electric field is uniform throughout the object being studied. In the EI imaging context, if the tissue is excited such that the electric field is uniform, the governing equations necessary for image reconstruction become linear, facilitating using efficient image reconstruction algorithms such as linear back projection (LBP). In that case the electric field uniformity is equivalent with governing equations linearity. An example of a metric for characterizing electric field uniformity is the normalized electric field standard deviation (NSTD). If the discretized form of the electric field is represented by E(xᵢ) where xᵢ represents a point in the space where the electric field is formed (i varies from 1 to N), the electric field NSTD is defined as follows in Equation 21:

$$NSTD = \frac{\sqrt{{\sum }_{i = 1}^{N} {( {E}_{i} - \overline{E} )}^{2}}}{\overline{E}} \qquad ( 21 )$$

An estimate of the value of electric field NSTD for the impedance scanner configuration (guard surface area 5 fold greater than and applied voltage equal to excitation cell surface area and applied voltage, respectively) used for producing permittivity tomography/3D images is no more than 20% (the smaller the value of this metric the higher the uniformity). An estimate of the value of electric field NSTD for the impedance scanner configuration (guard surface area approximately equal and applied voltage approximately equal to cell surface area and applied voltage, respectively) used for producing phase angle mammography/2D images is no more than 30%. NSTD estimates for a scanner configuration without any guard surrounding the excitation cell is often less than about 60%. These estimates are based on non-homogenous samples as may be found clinically. Comparison of uniformity may be easier with a standardized reference of a homogenous sample, such as a homogenous sample of adipose tissue. NSTD estimates using a homogenous sample of adipose tissue for the scanner configuration for permittivity tomography is less than 10%. NSTD estimates using a homogenous sample of adipose tissue for the scanner configuration for phase angle mammography is less than 5%. Taken together the NSTD estimates indicate that when the scanner configuration includes a guard for each excitation cell, deviation from uniformity can be less than 40%, less than 35%, less than 30%, less than 25%, less than 20%, less than 15%, less than 10%, less than 5% or less than any percentage therebetween. When the scanner is devoid of guards the deviation from uniformity is typically greater than 45% and typically less than 60%. The term substantially uniform is used to indicate deviation from uniformity of less than about 30%.

Deviation of an electric field communicating between a paired excitation cell and detector cell from an ideal uniform electric filed corresponding to 1D linearity may be characterized using a deviation metric Δc. Typically, average deviation of the electric field from linearity in the scanner configuration (larger guard) used for the permittivity tomography will be less than 25%. For example, average deviation may be less than 20%, 15%, 10%, 5% or less than any percentage therebetween. Similar calculations show that average deviation of the electric field from linearity in the scanner configuration (smaller guard) used for the phase angle mammography will be less than 30%. Estimates for deviation from linearity are in a similar range as estimates for deviation from uniformity. Typically, deviation from linearity is about 1.5 fold less than deviation from uniformity. The term substantially linear is used to indicate deviation from linearity of less than about 20%.

The impedance scanner can be used to provide two-dimensional (2D) images as well as three-dimensional (3D) images. An example of 2D imaging is mammography, while an example of 3D imaging is tomography. Permittivity, capacitance, and phase angle image reconstruction techniques may be useful in both 2D and 3D imaging. Linearity and uniformity of the impedance scanner clearly benefit 3D imaging as described above in examples of permittivity tomography. 2D imaging can accommodate larger deviation from linearity and larger deviation from uniformity. 2D imaging can accommodate deviations of 60%, 70%, 80% or even greater deviations from uniformity, particularly when quality of the image may not be critical, for example when the accuracy of the area of an inclusion is not the objective, but rather accurately detecting or imaging of presence or absence of an inclusion is the objective. Thus, 2D imaging can accommodate scanners that do not include a guard. However, a guard may be included to provide a benefit of improving the quality of the 2D image, for example improving the accuracy of the area of an inclusion as represented in the 2D image. Furthermore, constraining deviation from linearity and/or uniformity may benefit 2D imaging in providing greater confidence for image results, for example reducing occurrence of false negatives when an inclusion is located deep within a tissue sample. Linearity and/or uniformity are both positively correlated with reduced occurrence of artifacts.

Electrical impedance (EI) is a complex value that encompasses two major components of the material/sample (eg., tissue): resistance (R) and capacitance (C). By measuring the electrical impedance and phase angle, it is possible to delineate R and C. Furthermore, the resistance and capacitance are functions of the material intrinsic properties of the electrical conductivity (EP) and electrical permittivity (EP), respectively. As such, imaging the electrical impedance and phase angle enables image construction of R, C, EC and/or EP. In EI tomography (EIT), the measurements have to be performed by scanning the object under different rotating angles of the apparatus to allow reconstruction of a data set of EI and phase angle that can be processed to produce 3D images of EI, phase angle, EC and/or EP. In 2D EI imaging (e.g., EI mammography), data acquisition under a single angle is sufficient to produce 2D images of EI and/or phase angle, leading to 2D images of EC and/or EP.

As the scanner measures data in a form that allows for application of linear image reconstruction algorithms, image generation and image display may be achieved in real-time. Real-time imaging is readily supported for 2D imaging, and may even be achievable for 3D imaging.

The size of a cell typically ranges from 0.2 mm for high resolution imaging to 2 mm for low resolution imaging.

Distance between perimeters of neighboring cells is at least 0.05 mm, more typically at least 0.1 mm, generally ranging from about 0.1 mm to about 5 mm depending on requirements of a specific application. A minimum distance between perimeters of neighboring cells may be required to avoid significant disturbance of the 1D electric field uniformity. The minimum distance may vary depending on the manufacturing technique and process used to construct the impedance scanner plates. For example, in a printed circuit board (PCB) etching process, a cell is insulated from neighboring cells by a non-conductive gap of at least 0.1 mm.

The impedance scanner has been described as comprising a single line array of paired excitation and detector cells. The impedance scanner may readily be configured as a two-dimensional grid array of opposing paired excitation and detector cells.

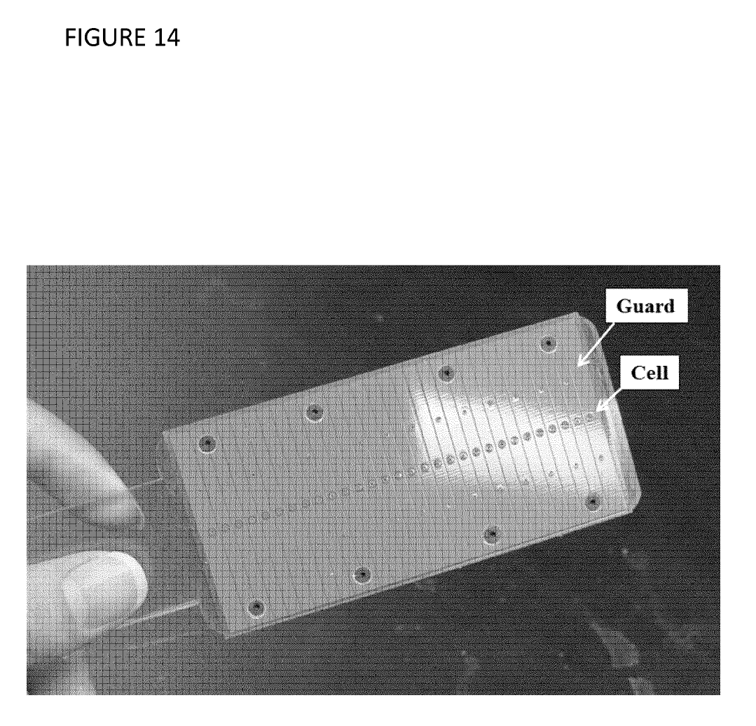

Each excitation cell may optionally be surrounded by a guard. The guard is a conductive material that may be the same material as the cell. The guard is electrically isolated from the cell by a non-conductive gap or non-conductive material, with the cell positioned within a central opening or aperture of the guard. The shape of the guard may be varied as desired including for example axi-symmetric (with respect to the center of the cell) shapes where portions of the guard are electrically isolated by one or more axi-symmetrically located non-conductive seaparators. FIG. 14 shows a circular excitation cell surrounded by a guard of conductive rectangular area defining a central circular opening or aperture for capturing the circular excitation cell and its surrounding non-conductive material area. The guard is excited with the same frequency and phase as the cell. The purpose of the guard is to focus the electric field line between corresponding excitation and detector cell pair and to minimize its bending. Each guard is electrically isolated from neighboring guards and each detector cell is electrically isolated from neighboring detector cells. This isolation is generated through the printed circuit board (PCB) process using non-conductive material where the gap between neighboring cells or neighboring guards range from 0.1 mm to 0.2 mm.

Guards can effect uniformity and linearity and reduce deviation from uniformity and linearity. The impact of the guard on reducing deviation from uniformity and linearity can be adjusted by changing the surface area of the guard and/or changing the voltage applied to the guard. Generally, keeping all other variables unchanged, an increase in surface area of the guard is positively correlated with uniformity. Similarly, in general, keeping all other variables unchanged, an increase in the voltage applied to the guard is positively correlated with uniformity. When a guard is used, the surface area of the guard is greater than half the surface area of its corresponding cell. Typically, the surface area of the guard will range from 0.5 fold to 10 fold the surface area of its corresponding cell. For example, the surface area of the guard may be greater than about 50%, 75%, 100%, 200%, 300%, 400%, 500% or greater than any percentage therebetween compared to the surface area of its corresponding cell. In certain examples, the surface area of the guard is about equal to or greater than the surface area of its corresponding cell. When a guard is used, voltage applied to the guard is greater than 5% of the voltage applied to its corresponding cell. Typically, the voltage applied to the guard will range from about 10% to 1000% of the voltage applied to its corresponding cell. For example, the voltage applied to the guard may be greater than 10%, 20%, 30%, 40%, 50%, 75%, 100%, 200%, 300%, 400%, 500%, 600%, 700%, 800% or greater than any percentage therebetween compared to the voltage applied to its corresponding cell. In certain examples, the voltage applied to the guard is about equal to or less than the voltage applied to its corresponding cell. Higher voltages would be used in conjunction with insulation covering the contacting surfaces.

Impedance scanners may be driven by an excitation signal to fire individual cells sequentially or to fire a plurality of cells simultaneously. In sequential firing, a sequence of firings occur where activation of each paired excitation cell and detector cell occurs while all other paired excitation cells and detector cells are off. Sequential firing, in the absence of a guard, may reduce linearity but may achieve higher contrast. Thus, firing cells sequentially, in the absence of a guard, may achieve better contrast but at the cost of solving nonlinear equations. Use of a guard may improve linearity for sequential firing. Furthermore, as shown in FIGS. 13A and 13B, simultaneous firing can achieve good linearity.

In the variant shown in FIG. 14 the material used to make the guard and cell arrays are the same. The cells and guards are made/etched from a thin copper layer laminated on an insulator pad/substrate (the insulator may be a suitable type of glass epoxy). The guards and cells are separated by a thin non-conductive gap on the copper layer of PCB through an etching process. The area which is located at the center is the cell and the area which surrounds the cell is the guard. In other examples, the guard and the excitation cell may be made of different conductive materials.

Both the cell and its surrounding guard are excited at the same time (simultaneously), thus the emission/propagation of the electric field from the cell and guard is simultaneous. In certain examples, the guard may be excited in advance of the excitation cell.

Typically, the guard and the cell are excited with the same frequency and phase. Amplitude of an applied excitation signal can be different between the guard and the excitation cell and maybe optimized to achieve a more focused beam. For example, a higher electric potential can be applied to the guard compared to the excitation cell in order to further focus the beam emitted by the excitation cell.

The purpose of the guard is to focus the electric field line between corresponding excitation and detection cell pair. Without wishing to be bound by theory, an electric field emitted from the guard may surround the electric field from the cell and constrain the electric field from the cell to a linear direction. Thus, the configuration of the guard may be modified to improve the 1D uniform electric field between an opposing excitation cell and detector cell pair.

The contacting surface of an excitation plate and/or a detector plate of the scanner may optionally be covered with an insulation material to form an insulation layer. The insulation layer was used in the scanner in the tomography experimental examples for imaging the tissue electrical permittivity. The insulation layer increases the resistance part of the impedance many fold, rendering measured impedance only sensitive to the capacitance part and enabling effective permittivity imaging. In the mammography experimental examples for imaging based on phase angle in addition to the other dielectric parameters, the insulation layer was removed in order to make the impedance measurement sensitive to the tissue resistance as well as its capacitance.

The data acquisition circuitry may be adapted to conventional circuitries, as long as it is capable of acquiring phase angle data and magnitude data of the impedance measurements. Accuracy of phase angle calculation is benefited by a high resolution data acquisition circuitry.

The impedance scanner system may be used for medical screening or medical imaging. A simplified variant of the impedance scanner system which does not require image reconstruction can be used effectively for medical screening. This is possible by visualizing the projected capacitance data, for example capacitance data from a single pass of a linear cell array. For medical imaging, the impedance scanner must be configured to capture sufficient data projections at different angles for image reconstruction.

Medical screening or medical imaging may be useful wherever existing imaging of tissues is performed, and may be particularly useful for tumour detection or imaging. For example, medical screening or medical imaging of a human female breast may be performed for detection of breast cancer.

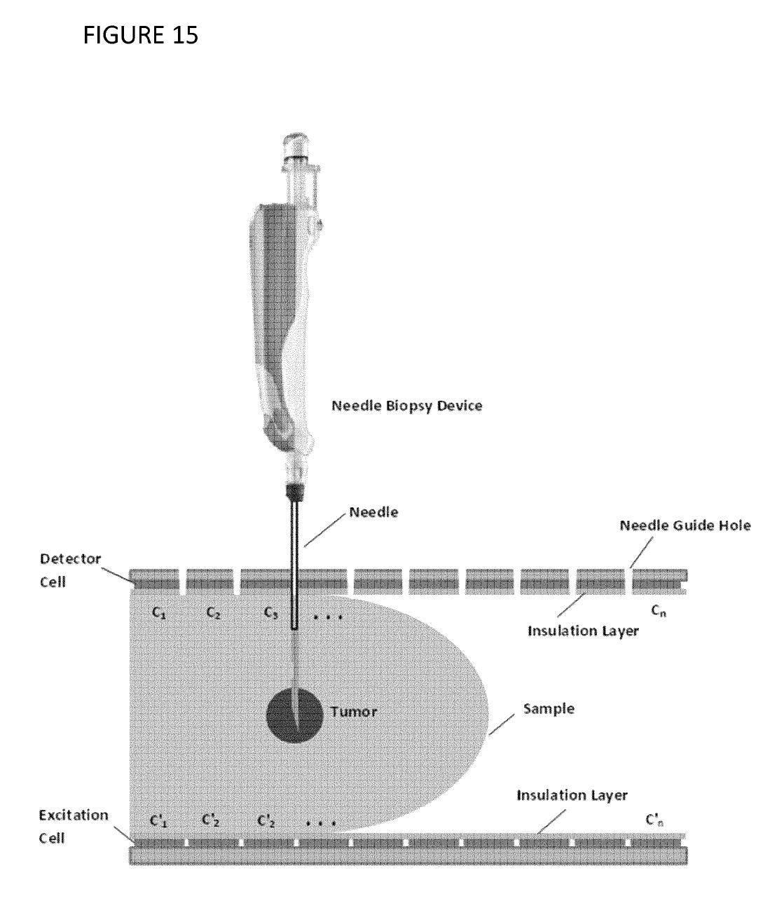

Medical imaging may be conducted for the purpose of image guided needle biopsy of a human female breast to accurately diagnose breast cancer. This is achieved by adding a grid with openings spaced in between excitation cells as illustrated in FIG. 15. The openings slidably receive a needle of a needle biopsy device. The grid openings provide a template for guiding needle insertions while guidance is provided by electrical permittivity tomography imaging achieved using the impedance scanner. This grid/template modification may also be used for insertions for therapeutic purposes.

The impedance scanner system may be used in conjunction with other imaging modalities such as x-ray, CT. and MM as may benefit a specific application.

Diagnostic methods of use of the scanner are contemplated including, for example, breast cancer screening/imaging, imaging/screening for edema (which includes for example pulmonary edema, pleural effusion and the like), and imaging/screening for detection or diagnosis of sepsis. Methods are configured on computer implement architecture and can include any combination of hardware and computer programmable code for activation of the scanner, making impedance measurement, storing impedance data in memory, processing impedance data with image reconstructions algorithms to generate an image, and displaying the image. Methods can also include computer programmable code algorithms for identifying an inclusion within an image including for example a tumor boundry, a tumor volume, an ablation boundary or an ablation volume.

Embodiments disclosed herein, or portions thereof, can be implemented by programming one or more computer systems or devices with computer-executable instructions embodied in a non-transitory computer-readable medium. When executed by a processor, these instructions operate to cause these computer systems and devices to perform one or more functions particular to embodiments disclosed herein. Programming techniques, computer languages, devices, and computer-readable media necessary to accomplish this are known in the art.

The computer readable medium is a data storage device that can store data, which can thereafter, be read by a computer system. Examples of a computer readable medium include read-only memory, random-access memory, CD-ROMs, magnetic tape, optical data storage devices and the like. The computer readable medium may be geographically localized or may be distributed over a network coupled computer system so that the computer readable code is stored and executed in a distributed fashion.

Computer-implementation of the system or method typically comprises a memory, an interface and a processor. The types and arrangements of memory, interface and processor may be varied according to implementations. For example, the interface may include a software interface that communicates with an end-user computing device through an Internet connection. The interface may also include a physical electronic device configured to receive requests or queries from a device sending digital and/or analog information. In other examples, the interface can include a physical electronic device configured to receive signals and/or data from an impedance scanner.

Any suitable processor type may be used depending on a specific implementation, including for example, a microprocessor, a programmable logic controller or a field programmable logic array. Moreover, any conventional computer architecture may be used for computer-implementation of the system or method including for example a memory, a mass storage device, a processor (CPU), a Read-Only Memory (ROM), and a Random-Access Memory (RAM) generally connected to a system bus of data-processing apparatus. Memory can be implemented as a ROM, RAM, a combination thereof, or simply a general memory unit. Software modules in the form of routines and/or subroutines for carrying out features of the system or method can be stored within memory and then retrieved and processed via processor to perform a particular task or function. Similarly, one or more method steps may be encoded as a program component, stored as executable instructions within memory and then retrieved and processed via a processor. A user input device, such as a keyboard, mouse, or another pointing device, can be connected to PCI (Peripheral Component Interconnect) bus. If desired, the software may provide an environment that represents programs, files, options, and so forth by means of graphically displayed icons, menus, and dialog boxes on a computer monitor screen.

Computer-implementation of the system or method may accommodate any type of end-user computing device including computing devices communicating over a networked connection. The computing device may display graphical interface elements for performing the various functions of the system or method. For example, the computing device may be a server, desktop, laptop, notebook, tablet, personal digital assistant (PDA), PDA phone or smartphone, and the like. The computing device may be implemented using any appropriate combination of hardware and/or software configured for wired and/or wireless communication. Communication can occur over a network, for example, where remote control of the system is desired.

If a networked connection is desired the system or method may accommodate any type of network. The network may be a single network or a combination of multiple networks. For example, the network may include the internet and/or one or more intranets, landline networks, wireless networks, and/or other appropriate types of communication networks. In another example, the network may comprise a wireless telecommunications network (e.g., cellular phone network) adapted to communicate with other communication networks, such as the Internet. For example, the network may comprise a computer network that makes use of a TCP/IP protocol (including protocols based on TCP/IP protocol, such as HTTP, HTTPS or FTP).

Embodiments described herein are intended for illustrative purposes without any intended loss of generality. Still further variants, modifications and combinations thereof are contemplated and will be recognized by the person of skill in the art. Accordingly, the foregoing detailed description is not intended to limit scope, applicability, or configuration of claimed subject matter.

1.-55. (canceled)

56. An electrical impedance scanner, comprising:
a first planar plate comprising a plurality of excitation cells;
a second planar plate comprising a plurality of detector cells;
the first planar plate held in spaced parallel relation to the second planar plate and defining a chamber therebetween;
the first and second planar plates arranged to align each excitation cell with a corresponding detector cell in a one-to-one paired relationship; and
a plurality of guards, each guard comprising a central opening for placing a single excitation cell, the guard electrically isolated from the excitation cell and from other guards, the guard made of a conductive material, and the guard is communicative with a voltage source.

57. The scanner of claim 56, wherein the excitation cell and the guard are made of the same material.

58. The scanner of claim 56, wherein the excitation cell and the guard are driven by an excitation signal of the same frequency and phase.

59. The scanner of claim 56, wherein the guard and the excitation cell are made of different conductive materials.

60. The scanner of claim 56, wherein activation of the plurality of excitation cells is coordinated by a first multiplexer and activation of the plurality of detector cells is coordinated by a second multiplexer.

61. The scanner of claim 60, further comprising a voltage source in communication with an input of the first multiplexer, the voltage source generating an excitation signal that can be modulated for amplitude, frequency or both amplitude and frequency.

62. The scanner of claim 60, further comprising data acquisition circuitry in electrical communication with an output of the second multiplexer, the data acquisition circuitry controlling measurement of magnitude, phase angle, or both magnitude and phase angle of impedance.

63. The scanner of claim 56, wherein each of the first and second planar plates comprise a contacting surface covered with an insulation material intended for abutting contact with a biological object.

64. The scanner of claim 56, wherein activation of each paired excitation cell and detector cell occurs while all other paired excitation cells and detector cells are off.

65. The scanner of claim 56, wherein activation of a plurality of paired excitation cells and detector cells occurs at the same time.

66. The scanner of claim 56, wherein the spaced relation between the first and second planar plates is adjustable to adjust the chamber volume.

67. The scanner of claim 62, further comprising an image reconstruction processor in electrical communication with the data acquisition circuitry, the image reconstruction processor configured to execute linear image reconstruction algorithms that include phase angle calculations.

68. The scanner of claim 56, further comprising an electric field communicating between a paired excitation cell and detector cell, and deviation of the electric field from uniformity is less than 40%.

69. The scanner of claim 56, further comprising an electric field communicating between a paired excitation cell and detector cell, and deviation of the electric field from linearity is less than 30%.

70. A computer-implemented method of electrical impedance imaging, comprising:
activating the scanner of claim 56 at a selected anatomical site;
making impedance measurements using the scanner to generate impedance data of the selected anatomical site;
communicating the impedance data to a processor;
processing the impedance data to generate an image.

71. The method of claim 70, wherein the selected anatomical site is a human female breast.

72. The method of claim 71, wherein the impedance measurements are made by generating an excitation signal having a frequency less than 10 KiloHertz.

73. The method of claim 71, wherein the impedance measurements are made by generating an excitation signal having a frequency less than 1 KiloHertz.

74. The method of claim 70, wherein the impedance data are processed using phase angle calculation.

75. The method of claim 71, further comprising identifying a tumour or an inclusion within the image.
